(19) United States
(12) Patent Application Publication
(10) Pub. No.: US 2020/0123258 A1
(43) Pub. Date: Apr. 23, 2020

(54) TARGETING B CELLS TO ENHANCE RESPONSE TO IMMUNE CHECKPOINT BLOCKADE

(71) Applicant: Board of Regents, The University of Texas System, Austin, TX (US)

(72) Inventors: Jennifer WARGO, Houston, TX (US); Sangeetha Meda REDDY, Houston, TX (US); Beth A. HELMINK, Houston, TX (US); Alexandria COGDILL, Houston, TX (US); Padmanee SHARMA, Houston, TX (US); James ALLISON, Houston, TX (US); Michael TETZLAFF, Houston, TX (US); Reetakshi ARORA, Houston, TX (US)

(21) Appl. No.: 16/661,867

(22) Filed: Oct. 23, 2019

Related U.S. Application Data
(60) Provisional application No. 62/749,576, filed on Oct. 23, 2018.

(51) Int. Cl. C07K 16/28; G01N 33/50; G16H 50/30

(52) U.S. Cl. CPC C07K 16/2818; G01N 33/5023; G16H 50/30; G01N 2800/60; G01N 2800/7028; G01N 2800/54; G01N 2333/7158; G01N 2333/70589; G01N 2800/52; G01N 2800/56; G01N 2333/70596

(57) ABSTRACT

Provided herein are methods for identifying a subject as a responder or non-responder to immune checkpoint blockade by detecting a B cell signature. Further provided herein are methods for treating cancer by administering immune checkpoint blockade therapy to a subject identified to have a B cell signature.

This application claims the benefit of U.S. Provisional Application No. 62/749,576, filed Oct. 23, 2018, the entirety of which is incorporated herein by reference.

### BACKGROUND

### 1. Field

The present invention relates generally to the fields of medicine and immunology. More particularly, it concerns methods for methods of treating cancer with immune checkpoint blockade.

### 2. Description of Related Art

Immunotherapy has afforded patients with melanoma and other cancers potential for long term survival, and there has been some insight into optimal biomarkers of therapeutic response. Significant progress has been made in this regard, with the identification of several validated biomarkers, particularly to immune checkpoint blockade (ICB). It is clear that cytotoxic T lymphocytes play a dominant role in response to ICB and other forms of immunotherapy; however, there is a growing appreciation of other components of the tumor microenvironment that may influence therapeutic response—including myeloid cells and other immune cell subsets. Tumor infiltrating B lymphocytes have been studied, though their functional role in cancer is incompletely understood—with some studies suggesting that they are tumor promoting while others show a positive association with improved cancer outcomes, particularly when they are found in association with tertiary lymphoid structures (TLS). However, their role in response to ICB remains unclear.

### SUMMARY

In certain embodiments, the present disclosure provides methods of treating cancer in a subject comprising administering an ICB therapy to the subject, wherein the subject has been determined to have a B cell signature. In further aspects, the B cell signature comprises increased expression of BANK1, CD19, CD22, CD79A, CR2, FCRL2, IGKC, MS4A1, and/or PAX5. In some aspects, the B cell signature comprises increased expression of BANK1, CD19, CD22, CD79A, CR2, FCRL2, IGKC, MS4A1, and PAX5.

In some aspects, the subject has a low percentage of tumor-infiltrating CD8⁺ T cells as dichotomized at a median value, such as a CD8 T cell score of less than 0 on MCP counter performed on gene expression profiling. In certain aspects, the B cell signature comprises a high density of tumor-infiltrating B cells. The high density may be a B cell lineage score of greater than −0.40 on MCP counter performed on gene expression profiling. The high density may be a tumor-infiltrating B cell density of at least 500 cells/mm² in the tumor, particularly at least 600, 700, 800, 900, 1000 or higher cells/mm² in the tumor. In particular aspects, the B cells are CD20+ and/or CD45±. In specific aspects, the B cells are positive for CD19 and/or MS4A1. In certain aspects, the B cell signature comprises high CXCL13 expression as compared to a control.

In certain aspects, the subject has a tumor comprising tertiary lymphoid structures (TLS). In particular aspects, the subject has a TLS density of at least 0.5 TLS/mm² in the tumor, such as at least 0.6, 0.7, 0.8, 0.9, 1 or higher TLS/mm² in the tumor. In specific aspects, the subject has an increased ratio of TLS per tumor area, such as a ratio of at least 0.25, 0.3, 0.4, 0.5, or higher TLS per tumor area.

In additional aspects, the method further comprises obtaining a sample from said subject. For example, the sample is blood, saliva, urine, or a tissue biopsy, specifically a tumor biopsy that may be fixed and paraffin-embedded (FFPE) or flash-frozen. In some aspects, the method further comprises isolating RNA from the sample. In certain aspects, the method further comprises determining the expression of B cell marker genes. In some aspects, determining the expression of B cell marker genes comprises preforming RT-PCR, a hybridization, transcriptome analysis, a Northern blot, a Western blot, RNA sequencing, or an ELISA.

In further aspects, the B cell signature comprises increased expression of BANK1, CD19, CD22, CD79A, CR2, FCRL2, IGKC, MS4A1, and/or PAX5. In some aspects, the B cell signature comprises increased expression of BANK1, CD19, CD22, CD79A, CR2, FCRL2, IGKC, MS4A1, and PAX5.

In some aspects, the B cell signature comprises co-localization of tumor-infiltrating B cells with CD4+, CD8+ and/or FoxP3+T lymphocytes. In certain aspects, the B cell signature comprises co-localization of tumor-infiltrating B cells with CD21 follicular dendritic cells.

In certain aspects, the subject has been previously administered ICB therapy. In other aspects, the subject has not been previously administered ICB therapy. The ICB therapy may be administered prior to and/or after surgery.

In some aspects, the ICB therapy is administered intravenously. In certain aspects, the ICB therapy comprises one or more inhibitors of CTLA-4, PD-1, PD-L1, PD-L2, LAG-3, BTLA, B7H3, B7H4, TIM3, KIR, or A2aR. In particular aspects, the ICB therapy comprises an anti-PD1 antibody and/or an anti-CTLA4 antibody. The anti-PD1 antibody may be nivolumab, pembrolizumab, pidillizumab, KEYTRUDA®, AMP-514, REGN2810, CT-011, BMS 936559, MPDL328OA or AMP-224. The anti-CTLA-4 antibody may be tremelimumab, YERVOY®, or ipilimumab. In particular aspects, the subject is administered nivolumab at a dose of 1 mg/kg and/or is administered ipilimumab at a dose of 3 mg/kg. In some aspects, the subject is administered ICB therapy more than once.

In further aspects, the subject is further administered or has been administered an anti-VEGF therapy, such as bevacizumab.

In additional aspects, the method further comprises administering at least one additional anti-cancer therapy. In some aspects, the anti-cancer therapy is a TLS targeted therapy, such as lymphotoxin receptor beta agonist. The anti-cancer therapy may be a CD40 agonist or regulatory T cells. In some aspects, the anti-cancer therapy is chemotherapy, immunotherapy, surgery, radiotherapy, or biotherapy. The anti-cancer therapy may be administered orally, intravenously, intraperitoneally, intratracheally, intratumorally, intramuscularly, endoscopically, intralesionally, percutaneously, subcutaneously, topically, regionally, or by direct injection or perfusion. In particular aspects, the ICB therapy and/or at one additional anti-cancer therapy is administered simultaneously. In some aspects, the ICB therapy is administered prior to the at least one additional anti-cancer therapy.

Another embodiment provides an in vitro method for detecting a B cell signature in a sample comprising obtaining a tumor sample from a subject diagnosed with cancer and detecting tumor-infiltrating B cells and/or TLS in said tumor sample, wherein an increased level of tumor-infiltrating B cells and/or TLS as compared to a control detects the B cell signature. In some aspects, the tumor sample is a tumor biopsy, such as a FFPE or flash-frozen tumor biopsy. In some aspects, detecting the B cell signature identifies the subject as ICB therapy sensitive.

In additional aspects, the method further comprises detecting co-localization of tumor-infiltrating B cells with CD4+, CD8+ and/or FoxP3+T lymphocytes. In some aspects, the method further comprises detecting co-localization of tumor-infiltrating B cells with CD21 follicular dendritic cells.

A further embodiment provides an in vitro method for detecting a B cell signature in a sample comprising obtaining a sample from a subject diagnosed with cancer; isolating RNA from the sample; and detecting an elevated expression of one or more B cell marker genes by performing RT-qPCR, microarray analysis, or RNA-sequencing on the isolated RNA, wherein elevated expression of one or more B cell marker genes as compared to a control identifies the B cell signature. In some aspects, the B cell marker genes comprise BANK1, CD19, CD22, CD79A, CR2, FCRL2, IGKC, MS4A1, and PAX5. In certain aspects, the sample is a tissue biopsy, fine needle aspirate, saliva, urine, or plasma. In some aspects, the tissue biopsy is further defined as FFPE tissue. In particular aspects, the tissue biopsy is further defined as a tumor biopsy. In some aspects, determining the expression of B cell marker genes comprises preforming RNA-sequencing. In certain aspects, detecting the B cell signature identifies the subject as ICB therapy sensitive.

In another embodiment, there is provided a method for treating cancer in a subject comprising administering ICB therapy to the subject, wherein the subject has been determined to have a tumor with TLS. In some aspects, the subject has a TLS density of at least 0.5 TLS/mm² in the tumor, such as at least 0.6, 0.7, 0.8, 0.9, 1 or higher TLS/mm² in the tumor. In certain aspects, the subject has an increased ratio of TLS per tumor area, such as a ratio of at least 0.25 TLS per tumor area, such as at least 0.5, 0.6, 0.7, 0.8, 0.9, 1 or higher TLS per tumor area.

In some aspects, the cancer is melanoma, such as high-risk resectable melanoma or metastatic melanoma. In certain aspects, the subject has been previously administered ICB therapy. In other aspects, the subject has not been previously administered ICB therapy. In some aspects, the ICB therapy is administered prior to and/or after surgery. In some aspects, the ICB therapy is administered intravenously. In specific aspects, the ICB therapy comprises one or more inhibitors of CTLA-4, PD-1, PD-L1, PD-L2, LAG-3, BTLA, B7H3, B7H4, TIM3, KIR, or A2aR. In some aspects, the ICB therapy comprises an anti-PD1 antibody and/or an anti-CTLA4 antibody. In certain aspects, the anti-PD1 antibody is nivolumab, pembrolizumab, pidillizumab, KEYTRUDA®, AMP-514, REGN2810, CT-011, BMS 936559, MPDL328OA or AMP-224. In some aspects, the anti-CTLA-4 antibody is tremelimumab, YERVOY®, or ipilimumab. In particular aspects, the subject is administered nivolumab at a dose of 1 mg/kg and/or is administered ipilimumab at a dose of 3 mg/kg. In some aspects, the subject is administered ICB therapy more than once.

In additional aspects, the method further comprises administering at least one additional anti-cancer therapy. In certain aspects, the subject is further administered or has been administered an anti-VEGF therapy, such as bevacizumab. In some aspects, the method further comprises administering a therapy to target TLS, such as a lymphotoxin receptor beta agonist. In some aspects, the method further comprises administering therapy to increase tumor-infiltrating B cells, such as inhibitors of immunosuppressive components of the tumor microenvironment, such as a CD40 agonist or T regulatory cells. In some aspects, the anti-cancer therapy is chemotherapy, immunotherapy, surgery, radiotherapy, or biotherapy. In certain aspects, the anti-cancer therapy is administered orally, intravenously, intraperitoneally, intratracheally, intratumorally, intramuscularly, endoscopically, intralesionally, percutaneously, subcutaneously, topically, regionally, or by direct injection or perfusion. In some aspects, the ICB therapy and/or at one additional anti-cancer therapy is administered simultaneously. In certain aspects, the ICB therapy is administered prior to the at least one additional anti-cancer therapy.

In another embodiment, there is provided a composition comprising an effective amount of an ICB therapy for use in the treatment of cancer in a subject, wherein the subject is determined to have a B cell signature. In some aspects, the B cell signature comprises increased tumor-infiltrating B cells as compared to a control and/or a tumor with TLS. In some aspects, the composition further comprises a lymphotoxin receptor beta agonist, CD40 agonist or regulatory T cells. In some aspects, the ICB therapy comprises an anti-PD1 antibody and/or an anti-CTLA-4 antibody.

In yet another embodiment, there is provided a method of predicting a response to an ICB therapy in a subject having a cancer comprising detecting increased tumor-infiltrating B cells and/or a TLS in a tumor sample obtained from said patient, wherein if the sample is positive for the presence of the increased tumor-infiltrating B cells and/or TLS, then the patient is predicted to have a favorable response to the ICB therapy. In some aspects, a favorable response to the ICB therapy comprises reduction in tumor size or burden, blocking of tumor growth, reduction in tumor-associated pain, reduction in cancer associated pathology, reduction in cancer associated symptoms, cancer non-progression, increased disease-free interval, increased time to progression, induction of remission, reduction of metastasis, or increased patient survival. In additional aspects, the method further comprises administering ICB therapy to said patient predicted to have a favorable response.

A further embodiment provides kit for determining the expression levels of B cell marker genes in a sample comprising primers that recognize one or more B cell marker genes selected from the group consisting of BANK1, CD19, CD22, CD79A, CR2, FCRL2, IGKC, MS4A1, and PAX5, or an array comprising said primers; and instructions for performing a method for determining the expression levels of said B cell marker genes.

In another embodiment, there is provided a kit for determining the level of B cell infiltrate and/or TLS in a tumor biopsy sample. The kit may comprise antibodies for B cell markers, such as CD20, CD45, CD19 and/or MS4A1. The kit may further comprise reagents for performing histological analysis of the tumor biopsy sample, such as to detect TLS within the sample.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawings(s) will be provided by the Office upon request and payment of the necessary fee.

The following drawings form part of the present specification and are included to further demonstrate certain aspects of the present disclosure. The present disclosure may be better understood by reference to one or more of these drawings in combination with the detailed description of specific embodiments presented herein.

FIGS. 1A-1D: Transcriptional analysis of tumor specimens from patients with high-risk resectable melanoma treated with pre-surgical immune checkpoint blockade (ICB). A, Supervised hierarchical clustering by response of melanoma tumor specimens at baseline of most differentially expressed genes (DEG) on RNA sequencing, with responder (R) defined as having a complete or partial response by RECIST 1.1 (n=9 NR and 7 R). B, Volcano plot depiction of DEG by response from same cohort as (A). C, Supervised clustering of melanoma tumor specimens by response at baseline (n=11 NR and 10 R), displaying MCP-counter scores (top panel), functional analysis (middle panel), and individual immune checkpoint related genes (bottom panel). D, Supervised clustering of melanoma tumor samples by response at on-treatment time-point (n=11 NR and 9 R). Comparisons were made using two-sided Mann-Whitney U tests.

FIG. 2: B cell signature is prognostic of improved survival in TCGA cutaneous melanoma cohort. Immune infiltrate is prognostic of improved disease-specific survival in TCGA cutaneous melanoma cohort. top, Unsupervised hierarchical analysis of TCGA SKCM RNA-seq data using MCP-counter scores identifies 3 melanoma immune clusters (MIC) with differential presence of individual immune cell types as indicated. q-value as shown. Bottom left, Kaplan-Meier estimates of overall survival (OS) of MIC groups. Bottom right, Kaplan-Meier estimates of OS by B-cell lineage scores shown by high and low groups dichotomized by median values. OS was defined as the time interval from date of accession for each sample to date of death or censoring from any cause. p-values calculated by log rank test.

FIG. 3: Immune infiltrate is not prognostic of improved disease-specific survival in TCGA clear cell renal cell carcinoma (RCC-TCGA KIRC) cohort. top, Unsupervised hierarchical analysis of TCGA KIRC RNA-seq data using MCP-counter scores identifies 3 immune clusters (IC) with differential presence of individual immune cell types as indicated. q-value as shown. Bottom left, Kaplan-Meier estimates of overall survival probability of IC groups. Bottom right, Kaplan-Meier estimates of overall survival probability by B-cell lineage scores shown by high and low groups dichotomized by median values. For both, OS was defined as the time interval from date of accession for each sample to date of death or censoring from any cause. p-values calculated by log rank test.

FIGS. 4A-4F: Tertiary lymphoid structures (TLS) containing B-cells, T-cells, and follicular dendritic cells are predictive of response to immune checkpoint blockade (ICB). A, Quantitation of CD20 cells by singlet immunohistochemistry and association with response to neoadjuvant ICB in resectable melanoma, with responders defined as having complete or partial response by RECIST 1.1 (n=11 NR and 9 R). Bars indicated median values, and individual data points in addition to interquartile ranges are shown. Comparisons were made using two-sided Mann-Whitney U tests. B, C, Density of TLS and ratio of tumor area occupied by TLS and correlation to treatment response (n=10 NR and 8 R, and 7 NR and 7 R, respectively). D, E, F, Representative case of responder to ipilimumab with nivolumab with TLS, associated hematoxylin and eosin slide, singlet stains, and characterization by multiplex immunofluorescence of TLS.

FIGS. 5A-5D: Analyses of B-cell receptor (BCR) clones and single cell analyses suggest active role for B-cells in anti-tumor immunity. A, Clonal counts for BCRs identified in patients with high-risk resectable melanoma treated with neoadjuvant ICB. Both the immunoglobulin heavy chain (IgH) and immunoglobulin light chain (IgL) are evaluated with responders (R) and non-responders (NR) as shown. All samples analyzed at baseline. B, tSNE plots demonstrating PBMC and intratumoral combined B cell populations from mass cytometric analyses in R vs NR (n=4 R and n=4 NR for PB and n=5 R and n=3 NR for tumor) from the neoadjuvant ICB trial in advanced melanoma patients. C, Intratumoral B cell phenotypes grouped by response. D, Quantitation of B-cell subtypes. Plots represent combined analyses of tumors ran simultaneously with the PBMC samples (n=5 R, n=3 NR) and include baseline and on-treatment samples.

FIGS. 6A-6C: MCP-counter results in melanoma and renal cell carcinoma (RCC) patients treated with pre-surgical immune checkpoint blockade (ICB). A, Supervised clustering by response of MCP-counter scores in on-treatment samples from a cohort of high-risk resectable melanoma patients treated with neoadjuvant ICB, with responder (R) defined as achieving a complete or partial response by RECIST 1.1 (n=11 NR and 9 R). B, Analysis shown by unsupervised hierarchical clustering of baseline and on-treatment samples from the neoadjuvant melanoma cohort. Unique clusters identified are indicated by shaded boxes on top row. C, Unsupervised hierarchical analysis shown for metastatic RCC patients. Response (PR, partial response) or non-response (PD, progressive disease) is measured by RECIST 1.1. Unique clusters identified are indicated by shaded boxes on top row.

FIGS. 7A-7B: Transcriptional analysis of tumor specimens from patients with metastatic renal cell carcinoma (RCC) treated with pre-surgical immune checkpoint blockade (ICB). A, Supervised hierarchical clustering by response of RCC tumor specimens at baseline of most differentially expressed genes (DEG) by microarray analysis, with response defined as having a partial response (PR) by RECIST 1.1 and non-response as having progressive disease (PD) (n=11 PD and 17 PR). A cut-off of gene expression fold change of or 0.5 and a FDR q-value of 0.05 was applied to select DEGs. B, Volcano plot depiction of DEG by response from same cohort.

FIGS. 8A-8B: Additional multiple IHC images of TLS. Multiplex IHC images from 6 more patients with (A) advanced melanoma treated with neoadjuvant immune checkpoint blockade or (B) metastatic renal cell carcinoma (RCC) treated with pre-surgical immune checkpoint blockade.

FIGS. 9A-9F: Tertiary lymphoid structures (TLS) containing B-cells, T-cells, and follicular dendritic cells are predictive of response to immune checkpoint blockade (ICB) in patients with renal cell carcinoma (RCC). A, Quantification of CD20 cells by singlet immunohistochemistry and association with response to neoadjuvant ICB in metastatic RCC, with responders defined as having partial response (PR) and non-responders as having progressive disease (PD) by RECIST 1.1. Density of TLS (B) and ratio of tumor area occupied by TLS (C) and correlation to treatment response. Bars indicate median and interquartile ranges are shown. Comparisons were made using two-sided Mann-Whitney U tests. D, E, F, Representative case of responder with TLS, associated hematoxylin and eosin slide, singlet stains, and characterization by multiplex immunofluorescence of TLS.

FIGS. 10A-10C: BCR analyses of intratumoral B-cells in patients with advanced melanoma prior to treatment with neoadjuvant checkpoint blockade. A, Clonal proportion for both immunoglobulin heavy chain (IgH) and immunoglobulin light chain (IgL) for baseline samples. Patients are grouped as responders (Rs) and non-responders (NRs) and identified as indicated. (B) Summed expression of Top 5 clones expressed in normalized read counts (B) and clonal diversity (C) for Rs as compared to NRs for both IgH and IgL. Box plot shows median and interquartile range. p-values calculated by two-sided Mann Whitney.

FIGS. 11A-11D: BCR analyses of intratumoral B-cells in patients with advanced melanoma following treatment with neoadjuvant checkpoint blockade. Clonal counts (A) and clonal proportions (B) for both immunoglobulin heavy chain (IgH) and immunoglobulin light chain (IgL) following treatment with immune checkpoint blockade. Patients are grouped as responders (Rs) and non-responders (NRs) and identified as indicated. Summed expression of Top 5 clones in normalized read counts (C) and clonal diversity (D) for Rs as compared to NRs for both IgH and IgL. All samples analyzed on treatment. Box plot shows median and interquartile range. p-values calculated by two-sided Mann Whitney.

FIGS. 12A-12B: Pathway analyses support differential B-cell phenotypes within the tumor. Gene set enrichment analysis for the bulk RNA-sequencing data comparing Rs and NRs to ICB at (A) baseline, and (B) on-treatment. The network-based pathway enrichment analysis was performed using the ReactomeFIViz, tool in Cytoscape using several biologic databases (K stands for KEGG database, N is NCBI database, R is reactome database, B is Biocarta database, and P is Panther database). All the pathways with FDR<0.05 were selected. The font size of pathways is based on the FDR significance—i.e. most significant pathways shown in larger fonts and least significant in smaller fonts.

FIGS. 13A-13B: Mass cytometry reveals significant differences in populations between responders and non-responder tumors. A, Pie charts representing makeup of individual tumor and PBMC samples for patients used in all analyses for mass cytometry. Matched patient samples are located directly beneath one another. Samples from patients with lymph node (LN) or non-LN metastases as indicated. Cell types as indicated. * indicates samples included in tSNE plots and pie charts. B, Scatter plots demonstrating quantification of different peripheral blood and intratumoral B cell phenotypes. Shown are mean and standard deviation. All samples are represented (for tumor, n=7R and n=3NR and, for PBMC, n=4R and n=4NR), p values calculated using one-sided Mann-Whitney.

FIG. 14: Surface expression of markers analyzed by mass cytometry. Individual phenographs for surface expression of each marker analyzed as indicated. This represents combined tumor and peripheral blood (PBMC) samples. These represent all samples ran together to avoid batch effect (8 tumor n=5R and n=3NR and 8 PBMC samples n=4R and n=4NR).

FIGS. 15A-15D: Mass cytometry demonstrates differences in peripheral blood and intratumoral B cells. A, Percentage of CD45+CD19+ cells by tissue type—peripheral blood versus tumor—that are positive for each of the surface markers indicated. B, Percentage of CD45+CD3+PD1+ cells identified in tumor versus peripheral blood. C, Percentage of CD45+CD19+ cells in tumor by response—responder (R) versus non-responder (NR)—that are positive for each of the surface markers indicated. D, Percentage of CD45+CD3+PD1+ cells identified within the tumor specimen. All samples are represented (for tumor, n=7R and n=3NR and, for PBMCs, n=4R and n=4NR). Error bars indicate mean and standard deviation. p-values were calculated by Mann-Whitney test.

### DESCRIPTION OF ILLUSTRATIVE EMBODIMENTS

Treatment with immune checkpoint blockade (ICB) has revolutionized cancer therapy, and efforts are underway to better understand therapeutic response. To date, total mutational load, T-cell markers, and PD-L1 have been identified as relevant biomarkers and T cells are typically considered to the major drivers of response, however there is a growing appreciation of the contribution of other immune subsets (including B-cells and tertiary lymphoid structures, TLS) in response to cancer therapy.

A neoadjuvant ICB phase II trial (PD-1 blockade monotherapy versus combined CTLA-4 and PD-1 blockade; NCT02519322) to assess the safety and feasibility of this treatment was conducted in in patients with high-risk resectable melanoma, and a B-cell signature was identified in responders to therapy via targeted expression profiling. Importantly, longitudinal tumor samples were taken in the context of therapy and molecular and immune profiling was performed in these samples to gain insight into mechanisms of therapeutic response and resistance. In these studies, known and novel biomarkers of response were identified, and targeted protein expression profiling (via Nanostring digital spatial profiling) revealed significantly higher expression of B-cell markers in baseline and on-treatment samples of responders to both single agent and combined ICB (Amaria, 2018).

To further study this, deep transcriptomic profiling was performed in baseline tumor samples from this cohort; demonstrating that the most differentially expressed genes in responders (Rs) as compared to non-responders (NRs) were B cell markers. Signatures were confirmed using an immune-based classification and were validated in a cohort of renal cell carcinoma patients treated with ICB, suggesting that B-cells and TLS may act in concert with T-cells in response to ICB across cancer types.

Informed by this data, RNAseq data was analyzed from the melanoma TCGA dataset and clustering of samples into 3 distinct immune subsets was identified with improved survival in the presence of high TLS/B-cell markers. Importantly, B-cell signatures were particularly predictive of survival and response in CD8 T-cell low cases. Histologic evaluation confirmed co-localization of B-cells in TLS, and single cell RNA sequencing analysis in an independent cohort of melanoma patients treated with ICB provided insight into B-cell phenotypes associated with improved clinical outcome. Together these data provide a novel predictive role for B-cells and TLS in response to ICB particularly in T-cell low tumors, and also provide provocative data regarding their potential contribution in response to cancer therapy.

Thus, in certain embodiments, the present disclosure provides methods for treating cancer by immune checkpoint blockade. The subjects may be assessed for their B cell and TLS signatures to predict response to ICB, particularly subjects with low CD8 T cell-infiltrated tumors. A high B cell and/or TLS signature can identify a subject that will response to ICB and have better overall survival. In addition, B cells can be targeted for enhancing outcome to therapy. These therapies include agents to stimulate tertiary lymphoid structures which contain B cell foci such as lymphotoxin beta receptor agonists or inhibitors of immunosuppressive components of the tumor microenvironment that prevent B cell proliferation and activation such as CD40 or T regulatory cells.

### I. DEFINITIONS

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more. The terms “about”, “substantially” and “approximately” mean, in general, the stated value plus or minus 5%.

As used herein, the terms “treat”, “treatment”, “treating”, and the like refer to the process of ameliorating, lessening, or otherwise mitigating the symptoms of a disease or condition in a subject by, for example, administering a therapeutic agent to the subject, or by performing a surgical, clinical, or other medical procedure on the subject.

As used herein, the terms “subject” or “patient” are used interchangeably herein to refer to an individual, e.g., a human or a non-human organism, such as a primate, a mammal, or a vertebrate.

The term “effective,” as that term is used in the specification and/or claims, means adequate to accomplish a desired, expected, or intended result. “Effective amount,” “therapeutically effective amount” or “pharmaceutically effective amount” when used in the context of treating a patient or subject with a compound means that amount of the compound which, when administered to a subject or patient for treating or preventing a disease, is an amount sufficient to affect such treatment or prevention of the disease.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

A “pharmaceutically acceptable carrier,” “drug carrier,” or simply “carrier” is a pharmaceutically acceptable substance formulated along with the active ingredient medication that is involved in carrying, delivering and/or transporting a chemical agent. Drug carriers may be used to improve the delivery and the effectiveness of drugs, including for example, controlled-release technology to modulate drug bioavailability, decrease drug metabolism, and/or reduce drug toxicity. Some drug carriers may increase the effectiveness of drug delivery to the specific target sites. Examples of carriers include: liposomes, microspheres (e.g., made of poly(lactic-co-glycolic) acid), albumin microspheres, synthetic polymers, nanofibers, protein-DNA complexes, protein conjugates, erythrocytes, virosomes, and dendrimers.

“Prognosis” refers to as a prediction of how a patient will progress, and whether there is a chance of recovery. “Cancer prognosis” generally refers to a forecast or prediction of the probable course or outcome of the cancer. As used herein, cancer prognosis includes the forecast or prediction of any one or more of the following: duration of survival of a patient susceptible to or diagnosed with a cancer, duration of recurrence-free survival, duration of progression-free survival of a patient susceptible to or diagnosed with a cancer, response rate in a group of patients susceptible to or diagnosed with a cancer, duration of response in a patient or a group of patients susceptible to or diagnosed with a cancer, and/or likelihood of metastasis and/or cancer progression in a patient susceptible to or diagnosed with a cancer. Prognosis also includes prediction of favorable survival following cancer treatments, such as a conventional cancer therapy.

As will be understood from context, a “risk” of a disease, disorder or condition comprises a likelihood that a particular individual will develop the disease, disorder, or condition.

As used herein, “overall survival” (OS) refers to the percentage of people in a study or treatment group who are still alive for a certain period of time after they were diagnosed with or started treatment for a disease, such as cancer. The overall survival rate is often stated as a five-year survival rate, which is the percentage of people in a study or treatment group who are alive five years after their diagnosis or the start of treatment.

The term “determining an expression level” as used herein means the application of a gene specific reagent such as a probe, primer or antibody and/or a method to a sample, for example a sample of the subject and/or a control sample, for ascertaining or measuring quantitatively, semi-quantitatively or qualitatively the amount of a gene or genes, for example the amount of mRNA. For example, a level of a gene can be determined by a number of methods including for example immunoassays including for example immunohistochemistry, ELISA, Western blot, immunoprecipitation and the like, where a biomarker detection agent such as an antibody for example, a labeled antibody, specifically binds the biomarker and permits for example relative or absolute ascertaining of the amount of polypeptide biomarker, hybridization and PCR protocols where a probe or primer or primer set are used to ascertain the amount of nucleic acid biomarker, including for example probe based and amplification based methods including for example microarray analysis, RT-PCR such as quantitative RT-PCR, serial analysis of gene expression (SAGE), Northern Blot, digital molecular barcoding technology, for example Nanostring:nCounter™ Analysis, and TaqMan quantitative PCR assays. Other methods of mRNA detection and quantification can be applied, such as mRNA in situ hybridization in formalin-fixed, paraffin-embedded (FFPE) tissue samples or cells. This technology is currently offered by the QuantiGene® ViewRNA (Affymetrix), which uses probe sets for each mRNA that bind specifically to an amplification system to amplify the hybridization signals; these amplified signals can be visualized using a standard fluorescence microscope or imaging system. This system for example can detect and measure transcript levels in heterogeneous samples; for example, if a sample has normal and tumor cells present in the same tissue section. As mentioned, TaqMan probe-based gene expression analysis (PCR-based) can also be used for measuring gene expression levels in tissue samples, and for example for measuring mRNA levels in FFPE samples. In brief, TaqMan probe-based assays utilize a probe that hybridizes specifically to the mRNA target. This probe contains a quencher dye and a reporter dye (fluorescent molecule) attached to each end, and fluorescence is emitted only when specific hybridization to the mRNA target occurs. During the amplification step, the exonuclease activity of the polymerase enzyme causes the quencher and the reporter dyes to be detached from the probe, and fluorescence emission can occur. This fluorescence emission is recorded and signals are measured by a detection system; these signal intensities are used to calculate the abundance of a given transcript (gene expression) in a sample.

The term “elevated expression” means an increase in mRNA production or protein production over that which is normally produced by non-cancerous cells. Non-cancerous cells for use in determining base-line expression levels can be obtained from cells surrounding a tumor, from other humans or from human cell lines. Any increase can have diagnostic value, but generally the mRNA or protein expression will be elevated at least about 3-fold, 5-fold, and in some cases up to about 100-fold over that found in non-cancerous cells.

The term “sample” as used herein includes any biological specimen obtained from a patient. Samples include, without limitation, whole blood, plasma, serum, red blood cells, white blood cells (e.g., peripheral blood mononuclear cells), ductal lavage fluid, nipple aspirate, lymph (e.g., disseminated tumor cells of the lymph node), bone marrow aspirate, saliva, urine, stool (i.e., feces), sputum, bronchial lavage fluid, tears, fine needle aspirate (e.g., harvested by fine needle aspiration that is directed to a target, such as a tumor, or is random sampling of normal cells, such as periareolar), any other bodily fluid, a tissue sample (e.g., tumor tissue) such as a biopsy of a tumor (e.g., needle biopsy) or a lymph node (e.g., sentinel lymph node biopsy), and cellular extracts thereof.

As used herein, the terms “control” and “standard” refer to a specific value that one can use to determine the value obtained from the sample. In one embodiment, a dataset may be obtained from samples from a group of subjects known to have a melanoma type or subtype. The expression data of the genes in the dataset can be used to create a control (standard) value that is used in testing samples from new subjects. In such an embodiment, the “control” or “standard” is a predetermined value for each gene or set of genes obtained from subjects with cancer subjects whose gene expression values and tumor types are known.

The term “altered” refers to a gene that is present at a detectably up-regulated or down-regulated level in a biological sample, e.g. tumor biopsy, from a patient that responds to ICB therapy, in comparison to a biological sample from a patient that is a non-responder to ICB therapy. The term includes increased or decreased expression in a sample from a patient with cancer due to transcription, post transcriptional processing, translation, post-translational processing, cellular localization (e.g, organelle, cytoplasm, nucleus, cell surface), and RNA and protein stability, as compared to a sample from a patient without cancer. Altered expression can be 10%, 20%, 30%, 40%, 50%, 60%, 70%, 80%, 90% or more in comparison to a control sample.

The terms “increased”, “elevated”, “overexpress”, “overexpression”, “overexpressed”, “up-regulate”, or “up-regulated” interchangeably refer to a gene that is present at a detectably greater level in a biological sample, e.g. tumor biopsy, from a patient that responds to ICB therapy, in comparison to a biological sample from a patient that is a non-responder to ICB therapy. The term includes overexpression in a sample from a patient with cancer due to transcription, post transcriptional processing, translation, post-translational processing, cellular localization (e.g, organelle, cytoplasm, nucleus, cell surface), and RNA and protein stability, as compared to a sample from a patient without cancer. Overexpression can be detected using conventional techniques for detecting mRNA (i.e., RT-PCR, PCR, hybridization) or proteins (i.e., ELISA, immunohistochemical techniques, mass spectroscopy, Luminex® xMAP technology). Overexpression can be 10%, 20%, 30%, 40%, 50%, 60%, 70%, 80%, 90% or more in comparison to a sample from a patient without cancer. In certain instances, overexpression is 1-fold, 2-fold, 3-fold, 4-fold 5, 6, 7, 8, 9, 10, or 15-fold or more higher levels of transcription or translation in comparison to a control sample.

A “biopsy” refers to the process of removing a tissue sample for diagnostic or prognostic evaluation, and to the tissue specimen itself. Any biopsy technique known in the art can be applied to the methods and compositions of the present invention. The biopsy technique applied will generally depend on the tissue type to be evaluated and the size and type of the tumor (i.e., solid or suspended (i.e., blood or ascites)), among other factors. Representative biopsy techniques include excisional biopsy, incisional biopsy, needle biopsy (e.g., core needle biopsy, fine-needle aspiration biopsy, etc.), surgical biopsy, and bone marrow biopsy. Biopsy techniques are discussed, for example, in Harrison's Principles of Internal Medicine, Kasper, et al., eds., 16th ed., 2005, Chapter 70, and throughout Part V. One skilled in the art will appreciate that biopsy techniques can be performed to identify cancerous and/or precancerous cells in a given tissue sample.

In the present text, a “good responder to a treatment”, also called a “responder” or “responsive” patient or in other words a patient who “benefits from” this treatment, refers to a patient who is affected with a cancer and who shows or will show a clinically significant relief in the cancer after receiving this treatment. Conversely, a “bad responder” or “non-responder” is one who does not or will not show a clinically significant relief in the cancer after receiving this treatment. The decreased response to treatment may be assessed according to the standards recognized in the art, such as immune-related response criteria (irRC), WHO or RECIST criteria.

### II. B CELL SIGNATURE

Certain embodiments of the present disclosure provide methods of identifying a subject with a B cell signature. The term “B cell signature” is used herein to refer to an increased number and/or density of tumor-infiltrating B cells in a subject with cancer and/or an altered expression of B cell markers. The B cells may be assessed by quantification of cells with CD20 and/or CD45 cell surface markers, such as by flow cytometry analysis, as well as the markers CD19 and MS4A1.

The B cell signature may be assessed by measuring the level of B cells, measuring the density of B cells, and/or by determining the expression of B cells markers in a sample, such as a patient sample. The patient may be diagnosed with or at risk for cancer, particularly melanoma. The sample may be a tumor sample, such as a biopsy. The biopsy may be FFPE or flash-frozen. The method may identify patients as responders or non-responders to ICB therapy and predict improved overall survival with ICB therapy if the patient sample is measured to have a B cell signature with increased B cells, increased B cell density, and/or increased expression of B cell markers.

In some embodiments, detecting expression of may comprise detecting levels of cDNA or RNA. Primers may be used in quantitative reverse transcriptase PCR and microarray methods for the amplification and detection of B cells markers or fragments thereof. In certain embodiments, gene expression can be analyzed using direct DNA expression in microarray, Sanger sequencing analysis, Northern blot, the NANOSTRING® technology, serial analysis of gene expression (SAGE), RNA-seq, tissue microarray, or protein expression with immunohistochemistry or western blot technique. Next generation sequencing methods can be performed using commercially available kits and instruments from companies such as the Life Technologies/Ion Torrent PGM or Proton, the Illumina HiSEQ or MiSEQ, and the Roche/454 next generation sequencing system.

The sample may be obtained from a subject, such as an animal or human. The sample may comprise tissue or fluid. A sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a sample is or comprises cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, or collection of body fluid (e.g., blood, lymph, feces). The sample may be obtained by processing (e.g., by removing one or more components of and/or by adding one or more agents to) a primary sample. For example, processing may comprise filtering using a semi-permeable membrane. Such a processed sample may comprise, for example nucleic acids extracted from a sample or obtained by subjecting a primary sample to techniques such as amplification, isolation and/or purification of certain components.

A person skilled in the art will appreciate that a number of detection agents can be used to determine the expression of the genes. For example, to detect RNA products of the biomarkers, probes, primers, complementary nucleotide sequences or nucleotide sequences that hybridize to the RNA products can be used. To detect protein products of the biomarkers, ligands or antibodies that specifically bind to the protein products can be used.

A reference or control sequence, sample, population, agent or individual is one that is sufficiently similar to a particular sequence, sample, population, agent or individual of interest to permit a relevant comparison (i.e., to be comparable). In some embodiments, information about a reference sample is obtained simultaneously with information about a particular sample. In some embodiments, information about a reference sample is historical. In some embodiments, information about a reference sample is stored for example in a computer-readable medium. In some embodiments, comparison of a particular sample of interest with a reference sample establishes identity with, similarity to, or difference of a particular sample of interest relative to a reference. In some embodiments, a reference for a marker is based on levels measured in an individual or population of individuals (e.g., an average across the population of 5, 10, 20 or more individuals) who do not present with symptoms of the disease in question (e.g., colorectal cancer). In some embodiments, a reference for a marker comprises a historical reference level for the marker from the individual being characterized.

In some embodiments, the subject is classified as having a B cell signature, and thus being a responder to ICB therapy, if the levels of one or more of BANK1, CD19, CD22, CD79A, CR2, FCRL2, IGKC, MS4A1, and PAX5 (i.e., genes that comprise the B cell score of the MCP counter) are increased as compared to a control level.

In some aspects, 1, 2, 3, 4, 5, 6, 7, 8, 9, 10, 11, 12, 13, 14, 15, 16, 17, 18, 19, 20, 25, 30, 40, 50, 75, 100, 120, or more of the B cell signature genes are used to determine the response to ICB therapy

In some of these aforementioned embodiments, a B cell signature may comprise B cell marker levels increased 1, 2, 3, 4, 5, 6, 7, 8, 9, 10, 15, 20, 25, 30, 35, 40, 45, 50, 55, 60, 65, 70, 75, 80, 85, 90, 95, 100, 1000% or more relative to a reference.

The subject may be further assessed for tertiary lymphoid structures (TLS). An ICB therapy responder may have increased quantity or density of TLS within the tumor. TLS may be qualified and quantified using both H&E and CD20 IHC staining. Structures may be identified as aggregates of lymphocytes having histologic features with analogous structures to that of lymphoid tissue with follicles, appearing in the tumor area (Pimenta et al., 2014). Criteria used for the quantification of TLS may include: 1) the total number of structures identified either within the tumoral area or in direct contact with the tumoral cells on the margin of the tumors (numbers of TLS/1 mm² area); and 2) a normalization of the total area occupied by the TLNs in relation of the total area of the tumor analyzed (ratio: area of TLS/area tumor+TLNs).

The B cell signature may be assessed by identifying co-localization of B cells with CD4+, CD8+ and/or FoxP3+T lymphocytes and/or CD21 dendritic cells. The co-localization may be assessed by immunofluorescence of a patient sample. For example, multiplex immunofluorescence assay may be performed for visualization of CD20, CD21, CD4, CD8, and/or FoxP3.

B. Isolation of RNA

Aspects of the present disclosure concern the isolation of RNA from a patient sample for use in determining the expression of B cell signature genes. The expression of other genes associated with B cell signature may also be assessed from the isolated RNA. The patient sample may blood, saliva, urine, or a tissue biopsy. The tissue biopsy may be a tumor biopsy that has been flash-frozen (e.g. in liquid nitrogen), FFPE, and/or preserved by an RNA stabilization agent (e.g., RNAlater). In some aspects, isolation is not necessary, and the assay directly utilizes RNA from within a homogenate of the tissue sample. In certain aspects the homogenate of FFPE tumor sample is enzymatically digested.

RNA may be isolated using techniques well known to those of skill in the art. Methods generally involve lysing the cells with a chaotropic (e.g., guanidinium isothiocyanate) and/or detergent (e.g., N-lauroyl sarcosine) prior to implementing processes for isolating particular populations of RNA. Chromatography is a process often used to separate or isolate nucleic acids from protein or from other nucleic acids. Such methods can involve electrophoresis with a gel matrix, filter columns, coated magnetic beads, alcohol precipitation, and/or other chromatography.

C. Expression Assessment

In certain aspects, methods of the present disclosure concern measuring expression of B cell markers gene(s). The expression information may be obtained by testing cancer samples by a lab, a technician, a device, or a clinician.

Expression levels of the gene(s) can be detected using any suitable means known in the art. For example, detection of gene expression can be accomplished by detecting nucleic acid molecules (such as RNA) using nucleic acid amplification methods (such as RT-PCR, droplet-based RT amplification, exon capture of RNA sequence library, next generation RNA sequencing), array analysis (such as microarray analysis), or hybridization methods (such as ribonuclease protection assay, bead-based assays, or Nanostring®. Detection of gene expression can also be accomplished using assays that detect the proteins encoded by the genes, including immunoassays (such as ELISA, Western blot, RIA assay, or protein arrays).

The pattern or signature of expression in each sample may then be used to generate a cancer prognosis or classification, such as predicting cancer survival or recurrence. The expression of one or more of genes could be assessed to predict or report prognosis or prescribe treatment options for cancer patients.

The expression of one or more genes may be measured by a variety of techniques that are well known in the art. Quantifying the levels of the messenger RNA (mRNA) of a gene may be used to measure the expression of the gene. Alternatively, quantifying the levels of the protein product of genes may be to measure the expression of the genes. Additional information regarding the methods discussed below may be found in Ausubel et al., (2003) Current Protocols in Molecular Biology, John Wiley &amp; Sons, New York, N.Y., or Sambrook et al. (1989) Molecular Cloning: A Laboratory Manual, Cold Spring Harbor Press, Cold Spring Harbor, N.Y. One skilled in the art will know which parameters may be manipulated to optimize detection of the mRNA or protein of interest.

A nucleic acid microarray may be used to quantify the differential expression of one or more genes. Microarray analysis may be performed using commercially available equipment, following manufacturer's protocols, such as by using the Affymetrix GeneChip® technology (Santa Clara, Calif.) or the Microarray System from Incyte (Fremont, Calif.). Typically, single-stranded nucleic acids (e.g., cDNAs or oligonucleotides) are plated, or arrayed, on a microchip substrate. The arrayed sequences are then hybridized with specific nucleic acid probes from the cells of interest. Fluorescently labeled cDNA probes may be generated through incorporation of fluorescently labeled deoxynucleotides by reverse transcription of RNA extracted from the cells of interest. Alternatively, the RNA may be amplified by in vitro transcription and labeled with a marker, such as biotin. The labeled probes are then hybridized to the immobilized nucleic acids on the microchip under highly stringent conditions. After stringent washing to remove the non-specifically bound probes, the chip is scanned by confocal laser microscopy or by another detection method, such as a CCD camera. The raw fluorescence intensity data in the hybridization files are generally preprocessed with a robust statistical normalization algorithm to generate expression values.

Quantitative real-time PCR (qRT-PCR) may also be used to measure the differential expression of one or more genes. In qRT-PCR, the RNA template is generally reverse transcribed into cDNA, which is then amplified via a PCR reaction. The amount of PCR product is followed cycle-by-cycle in real time, which allows for determination of the initial concentrations of mRNA. To measure the amount of PCR product, the reaction may be performed in the presence of a fluorescent dye, such as SYBR Green, which binds to double-stranded DNA. The reaction may also be performed with a fluorescent reporter probe that is specific for the DNA being amplified.

For example, extracted RNA can be reverse-transcribed using a GeneAmp® RNA PCR kit (Perkin Elmer, Calif., USA), following the manufacturer's instructions. In some embodiments, gene expression levels can be determined using a gene expression analysis technology that measure mRNA in solution. Methods of detecting gene expression are described for example in U.S. Patent Application Nos. US20140357660, and US20130259858; incorporated herein by reference. Examples of such gene expression analysis technologies include, but not limited to RNAscope™, RT-PCR, Nanostring®, QuantiGene®, gNPA®, HTG®, microarray, and sequencing. For example, methods of Nanostring use labeled reporter molecules, referred to as labeled “nanoreporters,” that are capable of binding individual target molecules. Through the nanoreporters' label codes, the binding of the nanoreporters to target molecules results in the identification of the target molecules. Methods of Nanostring are described in U.S. Pat. No. 7,473,767 (see also, Geiss et al., 2008). Methods may include the RainDance droplet amplification method such as described in U.S. Pat. No. 8,535,889, incorporated herein by reference. Sequencing may include exon capture, such as Illumina targeted sequencing after the generation of a tagged library for next generation sequencing (e.g. described in International Patent Application No. WO2013131962, incorporated herein by reference).

A non-limiting example of a fluorescent reporter probe is a TaqMan® probe (Applied Biosystems, Foster City, Calif.). The fluorescent reporter probe fluoresces when the quencher is removed during the PCR extension cycle. Multiplex qRT-PCR may be performed by using multiple gene-specific reporter probes, each of which contains a different fluorophore. Fluorescence values are recorded during each cycle and represent the amount of product amplified to that point in the amplification reaction. To minimize errors and reduce any sample-to-sample variation, qRT-PCR is typically performed using a reference standard. The ideal reference standard is expressed at a constant level among different tissues, and is unaffected by the experimental treatment. The system can include a thermocycler, laser, charge-coupled device (CCD) camera, and computer. The system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data.

To minimize errors and the effect of sample-to-sample variation, RT-PCR can be performed using an internal standard. The ideal internal standard is expressed at a constant level among different tissues, and is unaffected by an experimental treatment. RNAs commonly used to normalize patterns of gene expression are mRNAs for the housekeeping genes GAPDH, (3-actin, and 18S ribosomal RNA.

A variation of RT-PCR is real time quantitative RT-PCR, which measures PCR product accumulation through a dual-labeled fluorogenic probe (e.g., TAQMAN® probe). Real time PCR is compatible both with quantitative competitive PCR, where internal competitor for each target sequence is used for normalization, and with quantitative comparative PCR using a normalization gene contained within the sample, or a housekeeping gene for RT-PCR (see Heid et al., 1996). Quantitative PCR is also described in U.S. Pat. No. 5,538,848. Related probes and quantitative amplification procedures are described in U.S. Pat. Nos. 5,716,784 and 5,723,591. Instruments for carrying out quantitative PCR in microtiter plates are available from PE Applied Biosystems (Foster City, Calif.).

The steps of a representative protocol for quantitating gene expression level using fixed, paraffin-embedded tissues as the RNA source, including mRNA isolation, purification, primer extension and amplification are given in various published journal articles (see Godfrey et al., 2000; Specht et al., 2001). Briefly, a representative process starts with cutting about 10μιηthick sections of paraffin-embedded neoplasm tissue samples or adjacent non-cancerous tissue. The RNA is then extracted, and protein and DNA are removed. Alternatively, RNA is isolated directly from a neoplasm sample or other tissue sample. After analysis of the RNA concentration, RNA repair and/or amplification steps can be included, if necessary, and RNA is reverse transcribed using gene specific primers, followed by preparation of a tagged RNA sequencing library, and paired-end sequencing. In another example, the RNA is not reverse transcribed, but is directly hybridized to a specific template and then labeled with oligonucleotides and/or chemical or fluorescent color to be detected and counted by a laser.

In some embodiments, the PCR reaction is used in a “single-plex” PCR assay. “Single-plex” refers to a single assay that is not carried out simultaneously with any other assays. Single-plex assays include individual assays that are carried out sequentially.

In some embodiments, the PCR reaction is used in a “multiplex” PCR assay. The term “multiplex” refers to multiple assays that are carried out simultaneously, in which detection and analysis steps are generally performed in parallel. Within the context of the present disclosure, a multiplex assay will include the use of the primers, alone or in combination with additional primers to identify multiple genes simultaneously.

Immunohistochemical staining may also be used to measure the differential expression of one or more genes. This method enables the localization of a protein in the cells of a tissue section by interaction of the protein with a specific antibody. For this, the tissue may be fixed in formaldehyde or another suitable fixative, embedded in wax or plastic, and cut into thin sections (from about 0.1 mm to several mm thick) using a microtome. Alternatively, the tissue may be frozen and cut into thin sections using a cryostat. The sections of tissue may be arrayed onto and affixed to a solid surface (i.e., a tissue microarray). The sections of tissue are incubated with a primary antibody against the antigen of interest, followed by washes to remove the unbound antibodies. The primary antibody may be coupled to a detection system, or the primary antibody may be detected with a secondary antibody that is coupled to a detection system. The detection system may be a fluorophore or it may be an enzyme, such as horseradish peroxidase or alkaline phosphatase, which can convert a substrate into a colorimetric, fluorescent, or chemiluminescent product. The stained tissue sections are generally scanned under a microscope. Because a sample of tissue from a subject with cancer may be heterogeneous, i.e., some cells may be normal and other cells may be cancerous, the percentage of positively stained cells in the tissue may be determined. This measurement, along with a quantification of the intensity of staining, may be used to generate an expression value for the biomarker.

An enzyme-linked immunosorbent assay, or ELISA, may be used to measure the differential expression of one or more genes. There are many variations of an ELISA assay. All are based on the immobilization of an antigen or antibody on a solid surface, generally a microtiter plate. The original ELISA method comprises preparing a sample containing the biomarker proteins of interest, coating the wells of a microtiter plate with the sample, incubating each well with a primary antibody that recognizes a specific antigen, washing away the unbound antibody, and then detecting the antibody-antigen complexes. The antibody-antibody complexes may be detected directly. For this, the primary antibodies are conjugated to a detection system, such as an enzyme that produces a detectable product. The antibody-antibody complexes may be detected indirectly. For this, the primary antibody is detected by a secondary antibody that is conjugated to a detection system, as described above. The microtiter plate is then scanned and the raw intensity data may be converted into expression values using means known in the art.

An antibody microarray may also be used to measure the differential expression of one or more genes. For this, a plurality of antibodies is arrayed and covalently attached to the surface of the microarray or biochip. A protein extract containing the biomarker proteins of interest is generally labeled with a fluorescent dye.

The labeled gene protein(s) may be incubated with the antibody microarray. After washes to remove the unbound proteins, the microarray is scanned. The raw fluorescent intensity data may be converted into expression values using means known in the art.

Luminex multiplexing microspheres may also be used to measure the differential expression of a plurality of biomarkers. These microscopic polystyrene beads are internally color-coded with fluorescent dyes, such that each bead has a unique spectral signature (of which there are up to 100). Beads with the same signature are tagged with a specific oligonucleotide or specific antibody that will bind the target of interest (i.e., biomarker mRNA or protein, respectively). The target, in turn, is also tagged with a fluorescent reporter. Hence, there are two sources of color, one from the bead and the other from the reporter molecule on the target. The beads are then incubated with the sample containing the targets, of which up 100 may be detected in one well. The small size/surface area of the beads and the three-dimensional exposure of the beads to the targets allows for nearly solution-phase kinetics during the binding reaction. The captured targets are detected by high-tech fluidics based upon flow cytometry in which lasers excite the internal dyes that identify each bead and also any reporter dye captured during the assay. The data from the acquisition files may be converted into expression values using means known in the art.

In situ hybridization may also be used to measure the differential expression of a plurality of biomarkers. This method permits the localization of mRNAs of interest in the cells of a tissue section. For this method, the tissue may be frozen, or fixed and embedded, and then cut into thin sections, which are arrayed and affixed on a solid surface. The tissue sections are incubated with a labeled antisense probe that will hybridize with an mRNA of interest. The hybridization and washing steps are generally performed under highly stringent conditions. The probe may be labeled with a fluorophore or a small tag (such as biotin or digoxigenin) that may be detected by another protein or antibody, such that the labeled hybrid may be detected and visualized under a microscope. Multiple mRNAs may be detected simultaneously, provided each antisense probe has a distinguishable label. The hybridized tissue array is generally scanned under a microscope. Because a sample of tissue from a subject with cancer may be heterogeneous, i.e., some cells may be normal and other cells may be cancerous, the percentage of positively stained cells in the tissue may be determined. This measurement, along with a quantification of the intensity of staining, may be used to generate an expression value for each biomarker.

### III. IMMUNE CHECKPOINT BLOCKADE

In certain embodiments, the present disclosure provides methods of predicting response to immune checkpoint blockade therapy, such as by determining whether a subject has a B cell signature. Inhibitory immune checkpoint molecules that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors may target the PD-1 axis and/or CTLA-4.

The immune checkpoint inhibitors may be drugs such as small molecules, recombinant forms of ligand or receptors, or, antibodies, such as human antibodies. Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present invention. For example, it is known that lambrolizumab is also known under the alternative and equivalent names MK-3475 and pembrolizumab.

It is contemplated that any of the immune checkpoint inhibitors that are known in the art to stimulate immune responses may be used. This includes inhibitors that directly or indirectly stimulate or enhance antigen-specific T-lymphocytes. These immune checkpoint inhibitors include, without limitation, agents targeting immune checkpoint proteins and pathways involving PD-L2, LAG3, BTLA, B7H4 and TIM3. For example, LAG3 inhibitors known in the art include soluble LAG3 (IMP321, or LAG3-Ig disclosed in WO2009044273) as well as mouse or humanized antibodies blocking human LAG3 (e.g., IMP701 disclosed in WO2008132601), or fully human antibodies blocking human LAG3 (such as disclosed in EP 2320940). Another example is provided by the use of blocking agents towards BTLA, including without limitation antibodies blocking human BTLA interaction with its ligand (such as 4C7 disclosed in WO2011014438). Yet another example is provided by the use of agents neutralizing B7H4 including without limitation antibodies to human B7H4 (disclosed in WO 2013025779, and in WO2013067492) or soluble recombinant forms of B7H4 (such as disclosed in US20120177645). Yet another example is provided by agents neutralizing B7-H3, including without limitation antibodies neutralizing human B7-H3 (e.g. MGA271 disclosed as BRCA84D and derivatives in US 20120294796). Yet another example is provided by agents targeting TIM3, including without limitation antibodies targeting human TIM3 (e.g. as disclosed in WO 2013006490 A2 or the anti-human TIM3, blocking antibody F38-2E2 disclosed by Jones et al., J Exp Med. 2008; 205(12):2763-79).

B. PD-1 Axis Antagonists

In some embodiments, therapeutic targeting of PD-1 and other molecules which signal through interactions with PD-1, such as programmed death ligand 1 (PD-L1) and programmed death ligand 2 (PD-L2) is provided herein. For example, PD-1 axis binding antagonists include a PD-1 binding antagonist, a PDL1 binding antagonist and a PDL2 binding antagonist. Alternative names for “PD-1” include CD279 and SLEB2. Alternative names for “PDL1” include B7-H1, B7-4, CD274, and B7-H. Alternative names for “PDL2” include B7-DC, Btdc, and CD273. In some embodiments, PD-1, PDL1, and PDL2 are human PD-1, PDL1 and PDL2.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 axis antagonists for use in the methods provided herein are known in the art such as described in U.S. Patent Application No. US20140294898, US2014022021, and US20110008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 binding antagonists include Pidilizumab, also known as CT-011, MEDI0680, also known as AMP-514, and REGN2810.

In some embodiments, the immune checkpoint inhibitor is a PD-L1 antagonist such as Durvalumab, also known as MEDI4736, atezolizumab, also known as MPDL3280A, or avelumab, also known as MSB00010118C. In certain aspects, the immune checkpoint inhibitor is a PD-L2 antagonist such as rHIgM12B7. In some aspects, the immune checkpoint inhibitor is a LAG-3 antagonist such as, but not limited to, IMP321, and BMS-986016. The immune checkpoint inhibitor may be an adenosine A2a receptor (A2aR) antagonist such as PBF-509.

In some embodiments, the antibody described herein (such as an anti-PD-1 antibody, an anti-PDL1 antibody, or an anti-PDL2 antibody) further comprises a human or murine constant region. In a still further aspect, the human constant region is selected from the group consisting of IgG1, IgG2, IgG2, IgG3, and IgG4. In a still further specific aspect, the human constant region is IgG1. In a still further aspect, the murine constant region is selected from the group consisting of IgG1, IgG2A, IgG2B, and IgG3. In a still further specific aspect, the antibody has reduced or minimal effector function. In a still further specific aspect, the minimal effector function results from production in prokaryotic cells. In a still further specific aspect the minimal effector function results from an “effector-less Fc mutation” or aglycosylation.

Accordingly, an antibody used herein can be aglycosylated. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxy amino acid, most commonly serine or threonine, although 5-hydroxyproline or 5-hydroxy lysine may also be used. Removal of glycosylation sites form an antibody is conveniently accomplished by altering the amino acid sequence such that one of the above-described tripeptide sequences (for N-linked glycosylation sites) is removed. The alteration may be made by substitution of an asparagine, serine or threonine residue within the glycosylation site another amino acid residue (e.g., glycine, alanine or a conservative substitution).

The antibody or antigen binding fragment thereof, may be made using methods known in the art, for example, by a process comprising culturing a host cell containing nucleic acid encoding any of the previously described anti-PDL1, anti-PD-1, or anti-PDL2 antibodies or antigen-binding fragment in a form suitable for expression, under conditions suitable to produce such antibody or fragment, and recovering the antibody or fragment.

C. CTLA-4

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001014424, WO2000037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include soluble CTLA-4 ligands and receptors such as described in U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

D. Killer Immunoglobulin-Like Receptor (KIR)

Another immune checkpoint inhibitor for use in the present disclosure is an anti-KIR antibody. Anti-human-KIR antibodies (or VH/VL domains derived therefrom) suitable for use in the present methods can be generated using methods well known in the art.

Alternatively, art recognized anti-KIR antibodies can be used. The anti-KIR antibody can be cross-reactive with multiple inhibitory KIR receptors and potentiates the cytotoxicity of NK cells bearing one or more of these receptors. For example, the anti-KIR antibody may bind to each of KIR2D2DL1, KIR2DL2, and KIR2DL3, and potentiate NK cell activity by reducing, neutralizing and/or reversing inhibition of NK cell cytotoxicity mediated by any or all of these KIRs. In some aspects, the anti-KIR antibody does not bind KIR2DS4 and/or KIR2DS3. For example, monoclonal antibodies 1-7F9 (also known as IPH2101), 14F1, 1-6F1 and 1-6F5, described in WO 2006/003179, the teachings of which are hereby incorporated by reference, can be used. Antibodies that compete with any of these art-recognized antibodies for binding to KIR also can be used. Additional art-recognized anti-KIR antibodies which can be used include, for example, those disclosed in WO 2005/003168, WO 2005/009465, WO 2006/072625, WO 2006/072626, WO 2007/042573, WO 2008/084106, WO 2010/065939, WO 2012/071411 and WO 2012/160448.

An exemplary anti-KIR antibody is lirilumab (also referred to as BMS-986015 or IPH2102). In other embodiments, the anti-KIR antibody comprises the heavy and light chain complementarity determining regions (CDRs) or variable regions (VRs) of lirilumab. Accordingly, in one embodiment, the antibody comprises the CDR1, CDR2, and CDR3 domains of the heavy chain variable (VH) region of lirilumab, and the CDR1, CDR2 and CDR3 domains of the light chain variable (VL) region of lirilumab. In another embodiment, the antibody has at least about 90% variable region amino acid sequence identity with lirilumab.

### IV. METHODS OF USE

In some embodiments, the present disclosure provides methods for treating or delaying progression of cancer comprising administering an ICB therapy, such as to a subject identified to have a B cell signature. Biopsy samples from a patient undergoing immunotherapy can be assessed for the presence of increased B cell infiltration and/or tertiary lymphoid structures as biomarkers for improved responses to therapy. B cells and/or tertiary lymphoid structures can be used as therapeutic targets to enhance response of immune checkpoint blockade. Therapies increasing B cell infiltrates and tertiary lymphoid structures in combination with immunotherapies such as immune checkpoint blockade are also provided herein. Agents such as lymphotoxin receptor beta agonists that increase tertiary lymphoid structure formation can be combined with immune checkpoint blockade to improve treatment outcomes for cancer patients who are being treated with immunotherapy.

Tumors for which the present treatment methods are useful include any malignant cell type, such as those found in a solid tumor or a hematological tumor. Exemplary solid tumors can include, but are not limited to, a tumor of an organ selected from the group consisting of pancreas, colon, cecum, stomach, brain, head, neck, ovary, kidney, larynx, sarcoma, lung, bladder, melanoma, prostate, and breast. Exemplary hematological tumors include tumors of the bone marrow, T or B cell malignancies, leukemias, lymphomas, blastomas, myelomas, and the like. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); acute lymphoblastic leukemia (ALL); acute myeloid leukemia (AML); and chronic myeloblastic leukemia.

A. Combination Therapies

In certain embodiments, the methods provided herein further comprise a step of administering at least one additional therapeutic agent to the subject. All additional therapeutic agents disclosed herein will be administered to a subject according to good clinical practice for each specific composition or therapy, taking into account any potential toxicity, likely side effects, and any other relevant factors.

In certain embodiments, the additional therapy may be immunotherapy, radiation therapy, surgery (e.g., surgical resection of a tumor), chemotherapy, bone marrow transplantation, or a combination of the foregoing. The additional therapy may be targeted therapy. In certain embodiments, the additional therapy is administered before the primary treatment (i.e., as adjuvant therapy). In certain embodiments, the additional therapy is administered after the primary treatment (i.e., as neoadjuvant therapy.

In certain embodiments, the additional therapy comprises an immunotherapy. In certain embodiments, the immunotherapy comprises an immune checkpoint inhibitor.

An ICB therapy may be administered before, during, after, or in various combinations relative to an additional cancer therapy. The administrations may be in intervals ranging from concurrently to minutes to days to weeks. In embodiments where the ICB therapy is provided to a patient separately from an additional therapeutic agent, one would generally ensure that a significant period of time did not expire between the time of each delivery, such that the two compounds would still be able to exert an advantageously combined effect on the patient. In such instances, it is contemplated that one may provide a patient with the ICB therapy and the anti-cancer therapy within about 12 to 24 or 72 h of each other and, more particularly, within about 6-12 h of each other. In some situations, it may be desirable to extend the time period for treatment significantly where several days (2, 3, 4, 5, 6, or 7) to several weeks (1, 2, 3, 4, 5, 6, 7, or 8) lapse between respective administrations.

Various combinations may be employed. For the example below ICB therapy is “A” and an anti-cancer therapy is “B”:
A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

1. Chemotherapy

A wide variety of chemotherapeutic agents may be used in accordance with the present embodiments. Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammalI and calicheamicin omegaI1); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, farnesyl-protein tansferase inhibitors, transplatinum, and pharmaceutically acceptable salts, acids, or derivatives of any of the above.

2. Radiotherapy

Other factors that cause DNA damage and have been used extensively include what are commonly known as y-rays, X-rays, and/or the directed delivery of radioisotopes to tumor cells. Other forms of DNA damaging factors are also contemplated, such as microwaves, proton beam irradiation, and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

3. Immunotherapy

The skilled artisan will understand that immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells

Antibody—drug conjugates (ADCs) comprise monoclonal antibodies (MAbs) that are covalently linked to cell-killing drugs and may be used in combination therapies. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. Exemplary ADC drugs inlcude ADCETRIS® (brentuximab vedotin) and KADCYLA® (trastuzumab emtansine or T-DM1).

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, erb b2 and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies include immune adjuvants, e.g., Mycobacterium bovis, Plasmodium falciparum, dinitrochlorobenzene, and aromatic compounds); cytokine therapy, e.g., interferons α, β, and γ, IL-1, GM-CSF, and TNF; gene therapy, e.g., TNF, IL-1, IL-2, and p53; and monoclonal antibodies, e.g., anti-CD20, anti-ganglioside GM2, and anti-p185. It is contemplated that one or more anti-cancer therapies may be employed with the antibody therapies described herein.

4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin.

B. Pharmaceutical Compositions

In another aspect, provided herein are pharmaceutical compositions and formulations comprising 0 na ICB therapy and a pharmaceutically acceptable carrier.

Pharmaceutical compositions and formulations as described herein can be prepared by mixing the active ingredients (such as an antibody or a polypeptide) having the desired degree of purity with one or more optional pharmaceutically acceptable carriers (Remington's Pharmaceutical Sciences 22^{nd} edition, 2012), in the form of aqueous solutions, such as normal saline (e.g., 0.9%) and human serum albumin (e.g., 10%). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zinc-protein complexes); and/or non-ionic surfactants such as polyethylene glycol (PEG).

### V. KITS

In some embodiments, a kit that can include, for example, one or more media and components for the detection of a B cell signature is provided. Such components may comprise reagents, such as primers or antibodies, for the detection of B cell markers, B cell infiltration, and/or TLS in tumor samples. The reagent system may be packaged either in aqueous media or in lyophilized form, where appropriate. The container means of the kits will generally include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed. However, various combinations of components may be comprised in a vial. The components of the kit may be provided as dried powder(s). When reagents and/or components are provided as a dry powder, the powder can be reconstituted by the addition of a suitable solvent. It is envisioned that the solvent may also be provided in another container means. The kits also will typically include a means for containing the kit component(s) in close confinement for commercial sale. Such containers may include injection or blow molded plastic containers into which the desired vials are retained. The kit can also include instructions for use, such as in printed or electronic format, such as digital format.

### VI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—B Cells and Tertiary Lymphoid Structures (TLS) Contribute to Immune Checkpoint Blockade Response

A phase 2 clinical trial of neoadjuvant treatment with ICB was conducted in patients with high-risk resectable (clinical stage III or oligometastatic stage IV) melanoma to assess the safety and feasibility of this treatment in this patient population (NCT02519322) (Amaria et al., 2018). Importantly, longitudinal tumor samples were taken in the context of therapy, and molecular and immune profiling was performed to gain insight into mechanisms of therapeutic response and resistance. In these studies, known and novel biomarkers of response were identified, and targeted protein expression profiling (via Nanostring Digital Spatial Profiling) revealed significantly higher expression of B-cell markers in baseline and on-treatment samples of responders to ICB (Amaria et al., 2018).

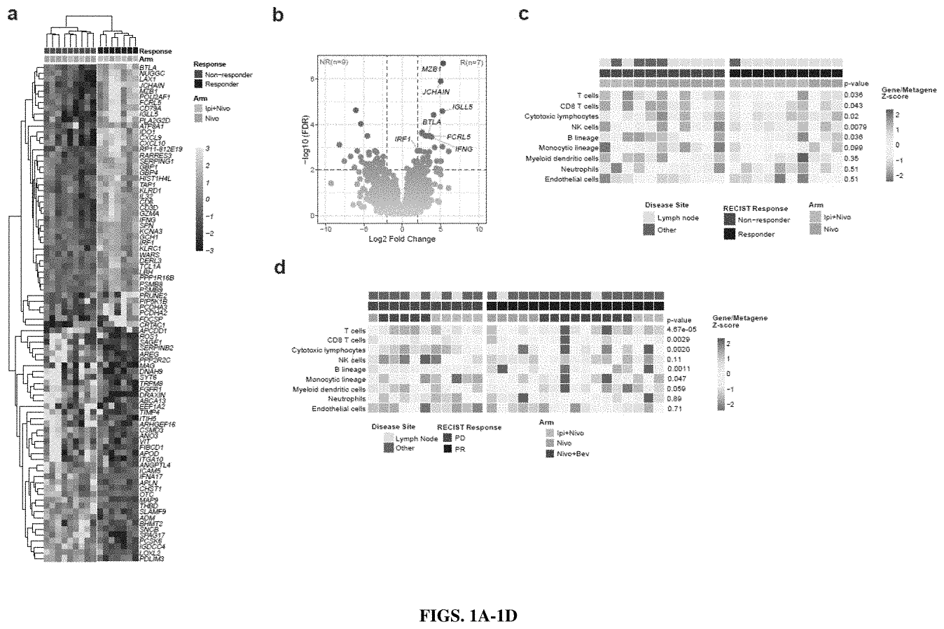

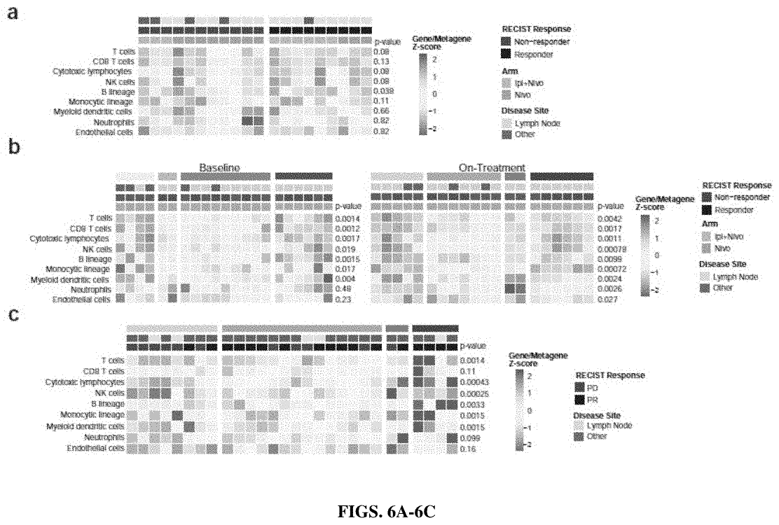

To gain a deeper understanding of potential mechanisms of therapeutic response to ICB in this cohort, RNA sequencing (RNAseq) was performed in longitudinal tumor samples from this patient cohort. In these studies, significantly higher expression of B-cell related genes such as MZB1, JCHAIN, and IGLL5 were observed in responders versus non-responders to ICB at baseline (p<0.001) with over-representation of these genes compared to T-cell and other immune markers (with evaluable tumors from 7R and 9NR) (FIGS. 1A-1B, Table 1). Additional genes that are expected to alter B-cell function were also significantly enriched in R vs NR, such as FCRL5, ID01, IFN-γ, and BTLA. Low tumor purity was observed in some samples, particularly in the context of an effective therapeutic response, limiting conventional analysis of RNAseq data. To address this, a more focused interrogation of the tumor immune microenvironment was performed using the MCP-counter method (Becht et al., 2016) on RNAseq data in baseline and on-treatment tumor samples—focusing more specifically on immune-related genes, allowing inclusion of samples with low tumor purity (10 R and 11 NR at baseline, 9 R and 11 NR on-treatment). In these analyses, enrichment of a B-cell signature was again observed in R versus NR at baseline and early on-treatment (p=0.036 and 0.038, respectively). Notably, these analyses included samples from patients with nodal and extra-nodal disease with no obvious contribution based on site of disease (FIGS. 1C, 6A-6B), suggesting that B-cell signatures were not merely related to the presence of these tumors within lymph nodes. B-cell signatures alone were predictive of response in univariable analyses (OR 2.6, p=0.02 for the trial, and OR 2.9, p=0.03 for combined melanoma cohorts), but not in multivariable analyses when considering other components of the immune cell infiltrate, suggesting that B-cells are likely acting in concert with other immune subsets and not acting in isolation; however these analyses were limited due to the low sample size (Tables 3 and 4).

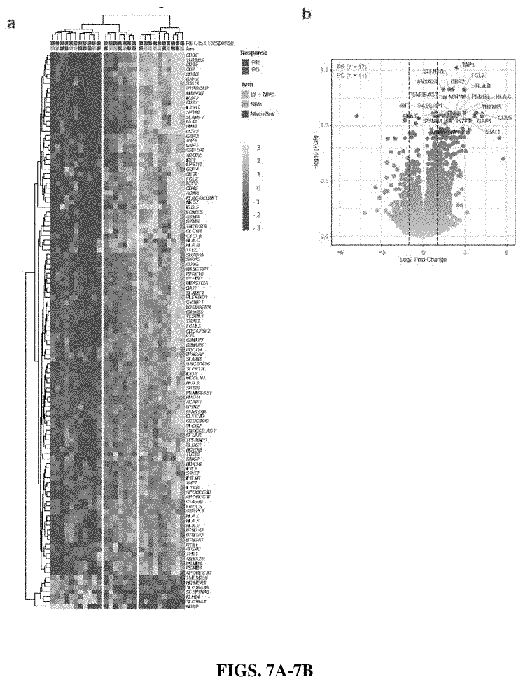

To evaluate the validity of these findings across additional cancer types, the expression of these immune cell gene expression signatures was next assessed in a pre-surgical ICB trial for patients with metastatic renal cell carcinoma (RCC) (NCT02210117, PD-1 blockade monotherapy versus combined CTLA-4 and PD-1 blockade versus combined PD-1 blockade and bevacizumab) (Table 2). Gene expression profiling by microarray and subsequent MCP-counter analysis of baseline tumor samples was performed, demonstrating significantly higher expression of B-cell related genes in R vs NR to therapy (p=0.0011, n=17R and 11 NR) (FIGS. 1D, 6C, 7). As in the case of melanoma, B-cell signatures were predictive of response in univariable analysis in the RCC cohort (OR 61.2, p=0.05) but not multivariable analysis, again suggesting cooperative function with other immune subsets; however, sample size was again quite limited (Table 5).

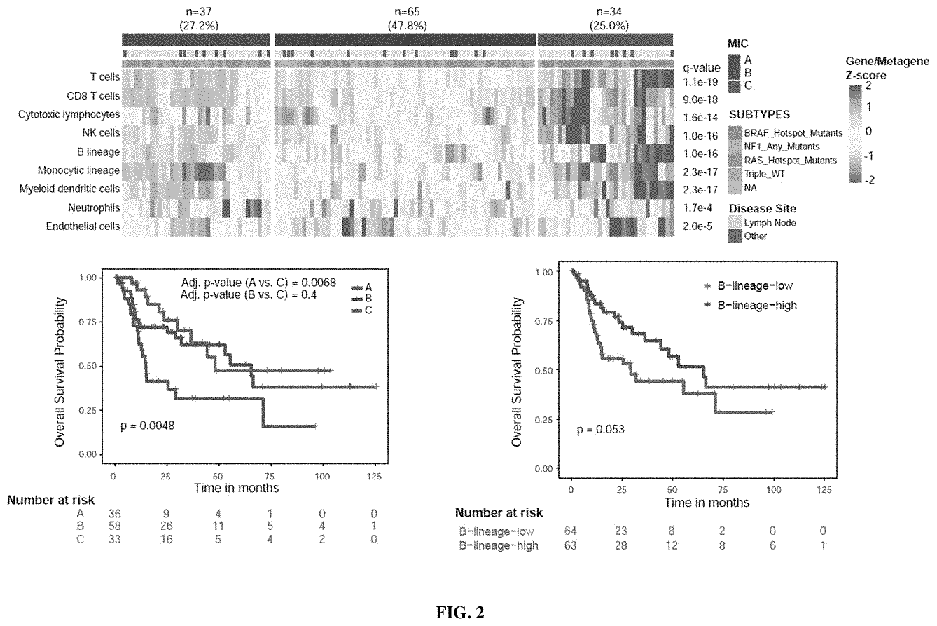

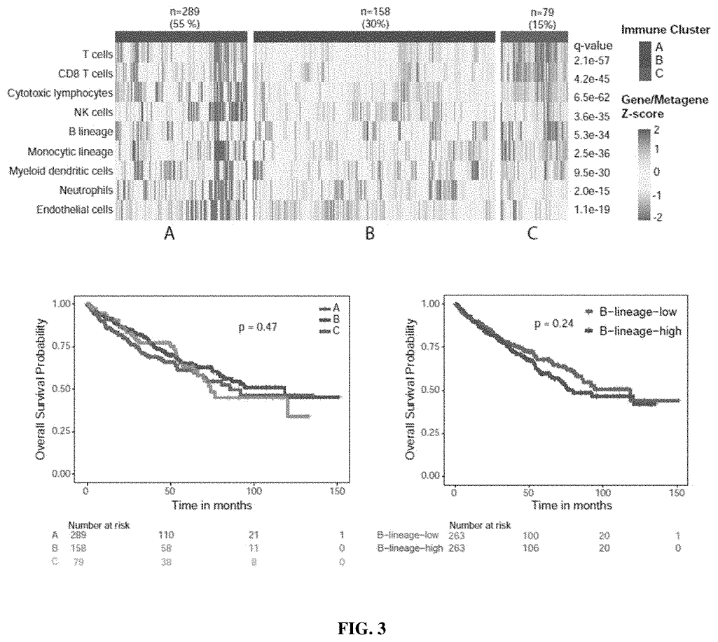

Based on these data along with existing data regarding a potential prognostic role for TLS in melanoma and other cancer types outside the context of treatment with ICB, the expression of these immune related genes was next assessed in cutaneous melanoma from The Cancer Genome Atlas platform (TCGA-SKCM, n=136) (Cancer Genome Atlas Network, 2015). To do this, the MCP-counter algorithm was applied to available RNAseq data from a subset of patients with non-recurrent Stage III disease (regional lymph node or regional subcutaneous metastases), as these were most comparable to the clinical cohort. In these studies, 3 distinct melanoma immune clusters (MICs), were identified with significantly higher expression of B-cells in cluster C versus cluster A (p<0.0001) and cluster B (p<0.0001) (FIG. 2I). Notably, there was no clear association of MIC with known genomic subtypes of melanoma (BRAF, NRAS, NF1, triple WT) or disease site (nodal versus non-nodal) (FIG. 2I). Importantly, survival analyses revealed that cases in cluster C had significantly improved overall survival (OS) compared to cluster A (p=0.0068) (FIG. 2I). To assess the association with B-cell signatures specifically, OS was next compared between B-cell lineage high versus low demonstrating prolonged survival in patients with B-cell lineage high versus to B-cell lineage low tumors (p=0.053) (FIG. 2I). Furthermore, univariable Cox Proportional Hazards modeling demonstrated that tumors with low B-cell infiltrate had significantly increased risk of death (HR is 1.7 for B-cell low, p=0.05) in comparison to B-cell high group (Table 6). Similar analyses were performed to assess the expression of immune-related genes in clear cell RCC from the TCGA (TCGA-KIRC, n=526). In these studies, similar immune clusters were observed; however, immune infiltrate was not associated with survival in these patients (p=0.24) (FIG. 3H), possibly owing to the heterogeneous nature of this disease and other driving mechanisms of patient outcomes.

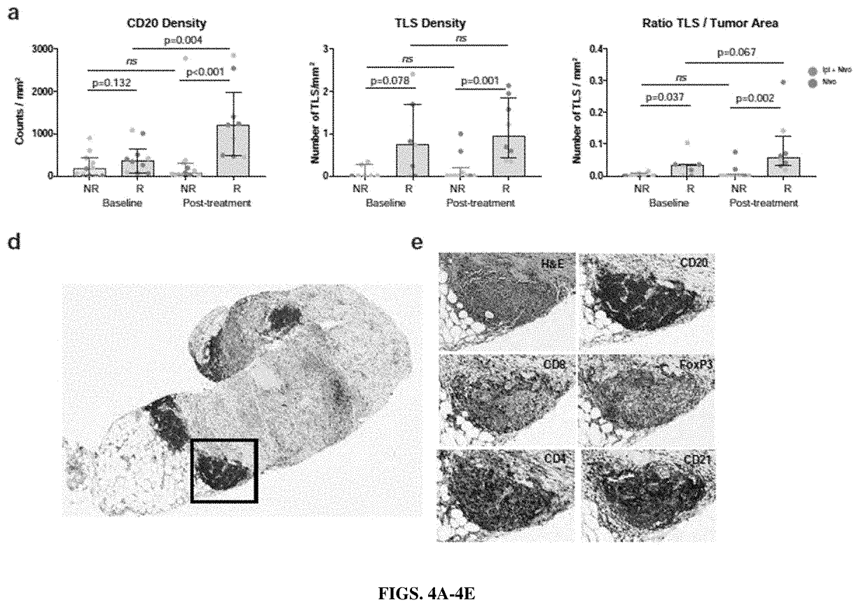

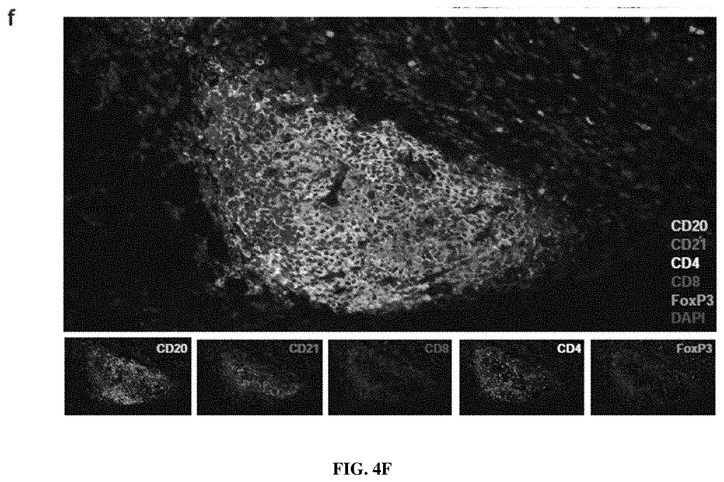

Based on these insights from gene expression profiling data, tumor samples were next assessed histologically to gain insight into the density and distribution of B-cells as well as theirrelationship to TLS in patients treated with neoadjuvant ICB. The density of CD20+ B-cells, TLS, and ratio of TLS to tumor area was higher in R versus NR in the neoadjuvant melanoma cohort, particularly in early on-treatment samples (p=0.0008, p=0.001, p=0.002 respectively), though statistical significance was not reached for all of these markers in baseline samples (p=0.132, p=0.078, p=0.037, respectively) (FIG. 4A), which is in line with the prior published work suggesting that assessment of early on-treatment immune infiltrate is far more predictive of response to ICB than assessment of pre-treatment samples (Chen et al., 2016).

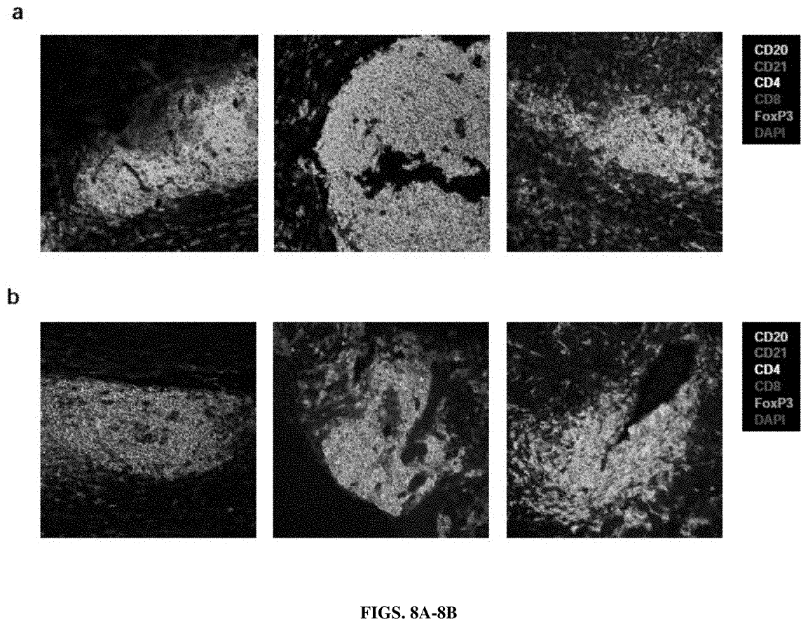

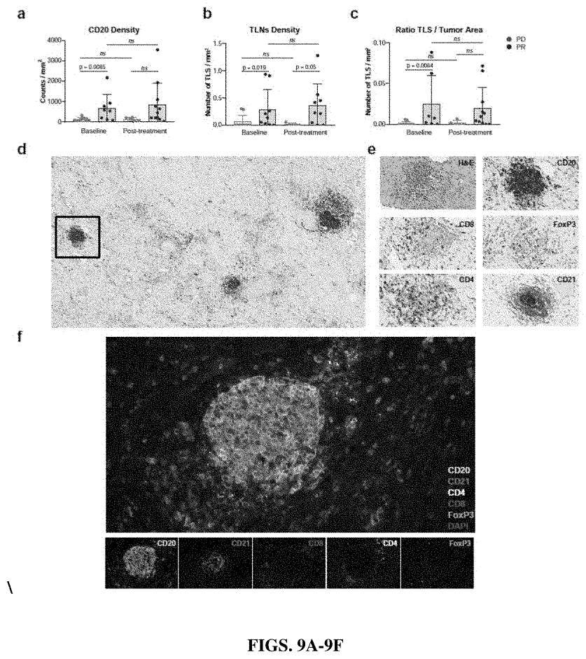

Importantly, architectural analysis identified that CD20+ B-cells were localized in TLS within tumors of Rs with co-localization of CD20⁺ B-cells with CD4⁺, CD8⁺, and FoxP3⁺ T lymphocytes. Co-localization with CD21⁺ follicular dendritic cells and MECA79⁺ high endothelial venules (HEV) was also demonstrated (FIGS. 4D-F, 8A). The vast majority of evaluated TLS in these patients represented mature secondary-follicle like TLS, as indicated by the presence of both CD21+ follicular dendritic cells and CD23⁺ germinal center B-cells (Posch et al., 2018) (FIGS. 4D-F, 8A). Analogous immunohistochemical findings were observed in the cohort of RCC patients treated with pre-surgical ICB with increased CD20+ cell infiltration and TLS density associated with response (FIG. 9A-C); these TLS are morphologically similar to those found in melanoma (FIGS. 8B, 9D-F).

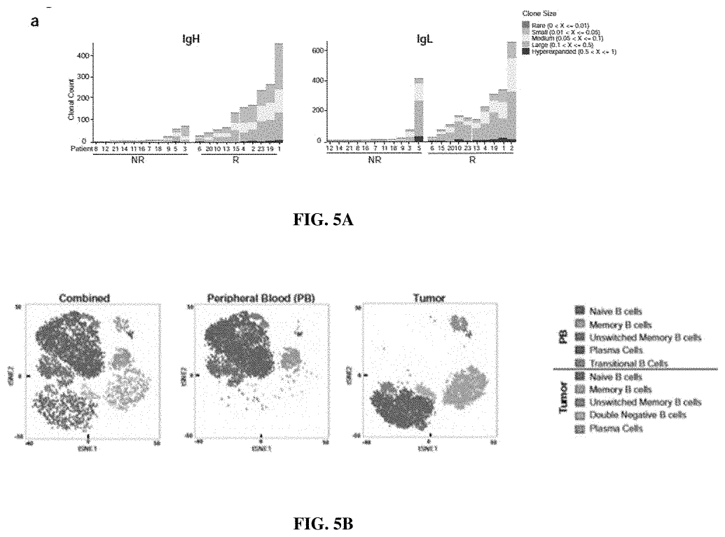

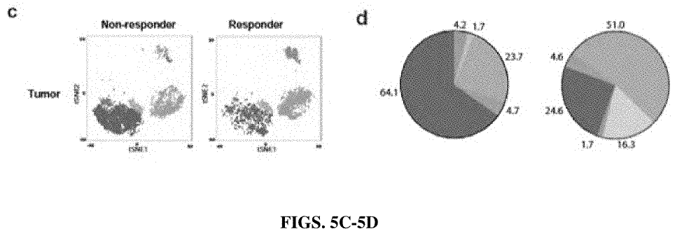

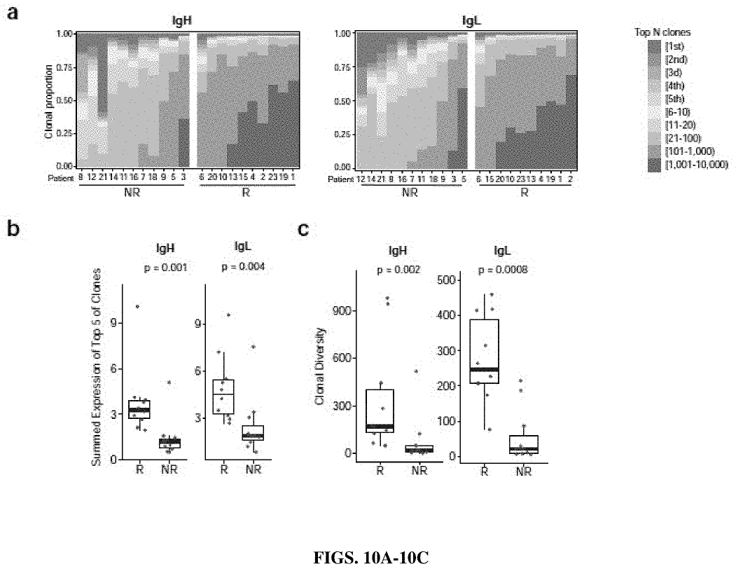

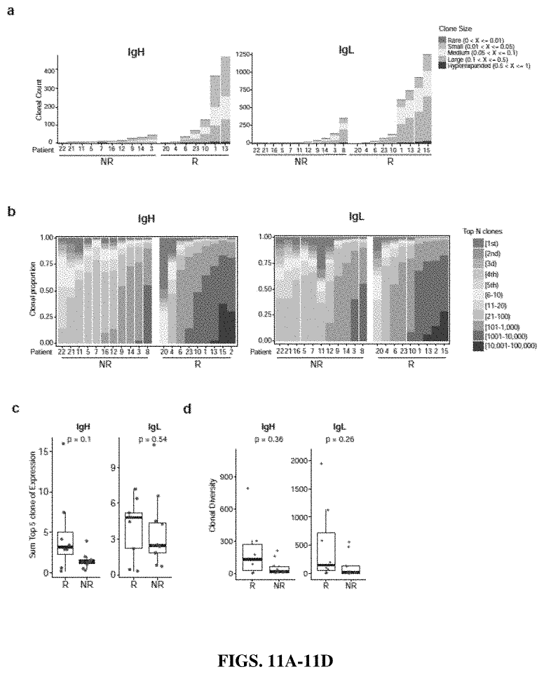

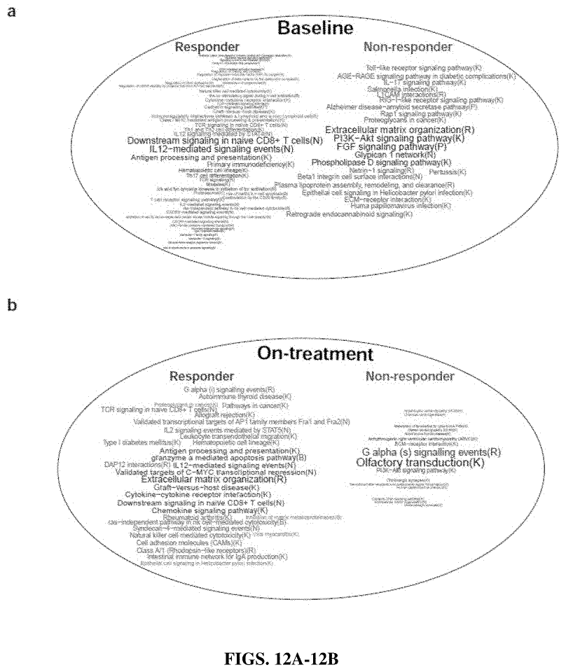

Next, several more in-depth analyses were performed to gain insight into the phenotype and function of the infiltrating B-cells, and how they might be contributing to responses to ICB. Reasoning that differences in clonotypes of B-cell receptors (BCRs) between Rs and NRs would be indicative of an antitumor B-cell response, RNAseq data was probed for BCR sequences using the modified TRUST algorithm. In these studies, significantly increased clonal counts were identified for both immunoglobulin heavy chain (IgH) and immunoglobulin light chain (IgL) (p=0.001 and p=0.004, respectively) and increased BCR diversity in Rs as compared to NRs (p=0.002 and p=0.0008) suggesting an active role for B-cells in anti-tumor immunity (FIGS. 5, 10, 11). Pathway analysis was also performed on bulk RNA sequencing data from the clinical trial cohort, revealing increased immune signaling pathways in Rs as compared to NRs including TCR signaling, MHC-mediated antigen processing and presentation, Th1- and Th2-cell differentiation, and co-stimulatory signaling associated with T-cell signaling (FIG. 12, Tables 7 and 8).

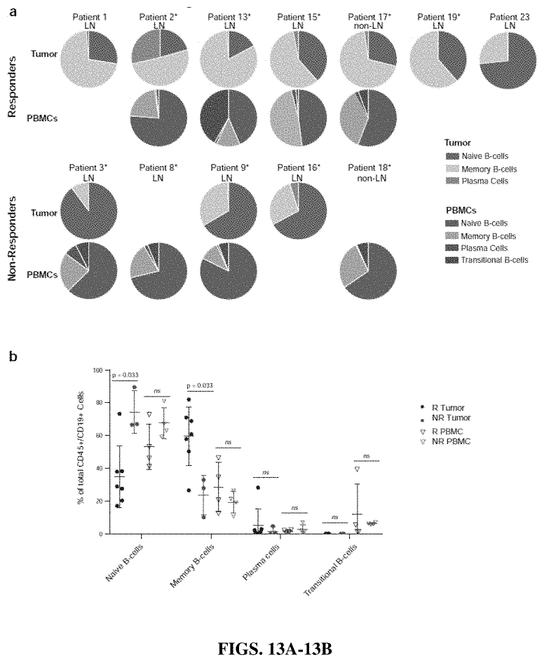

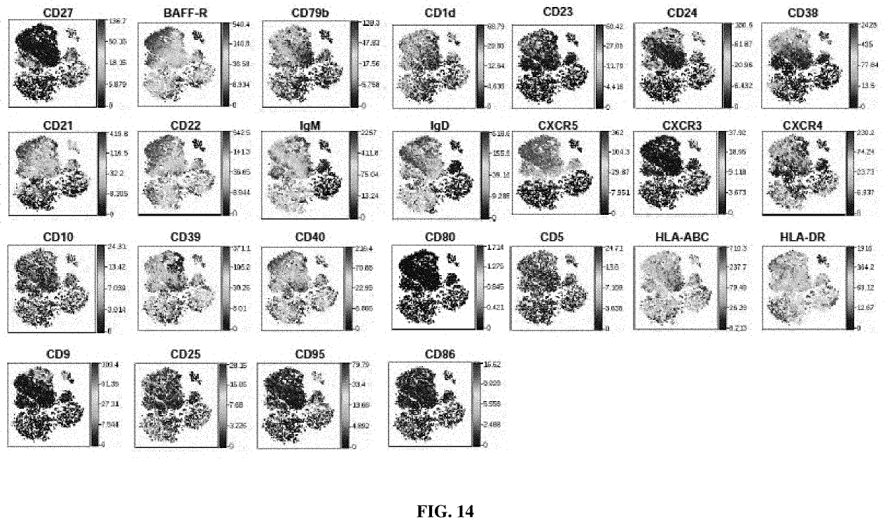

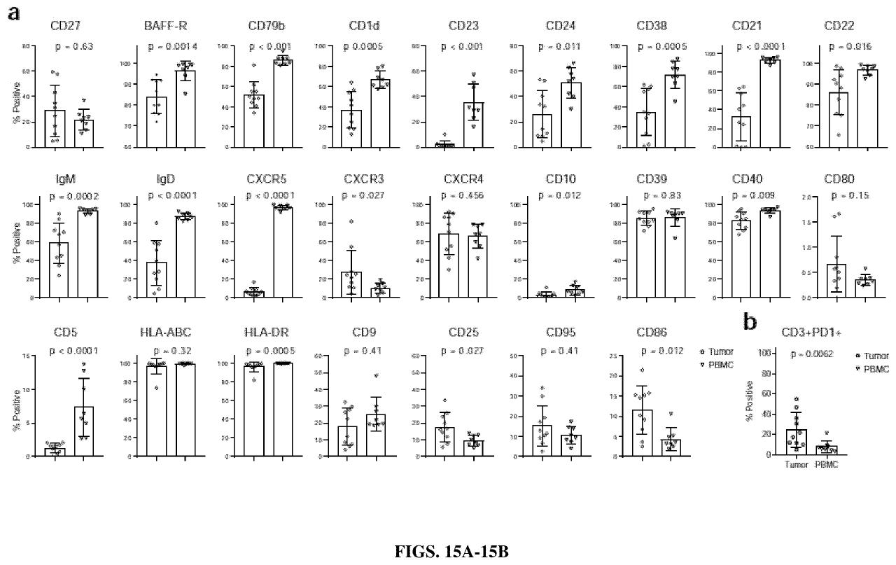

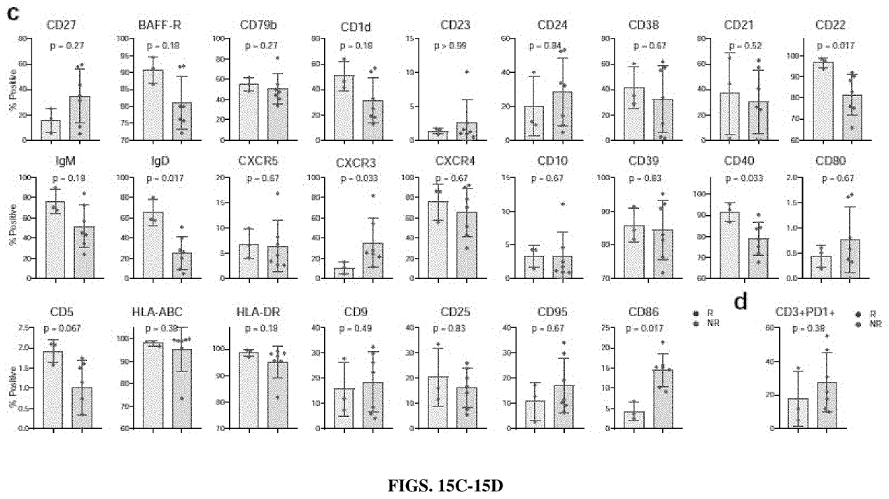

To gain additional insight into the potential functional role of B-cells in response to ICB, mass cytometry (CyTOF) was performed in evaluable tumor and peripheral blood (PB) samples (n=7 R and n=3 NR for tumor and n=4 R and n=4 NR for PB from the neoadjuvant ICB trial). Sample size was somewhat limited due to the amount of tumor available given prioritization for other studies as well as tumor viability. Notably, these analyses include patients with nodal and non-nodal metastases (FIG. 13A, Table 10). The differences were first assessed between intra-tumoral B-cells and those in the peripheral blood of patients. In these studies, unique clusters of CD45+CD19+(B-cell) populations including naïve (CD19⁺, CD27⁻, IgD⁺), transitional (CD19⁺, CD24⁺⁺, CD38⁺⁺, CD10⁺, CD27⁻, IgD⁺), unswitched and switched memory (CD19⁺, CD27⁺, IgD^{+/-}), double-negative (CD19⁺, CD27⁻, IgD⁻), and plasma(like) cell (CD19⁺, CD20⁻, CD22⁻, CD38⁺⁺, CD27⁺⁺) populations were demonstrated in peripheral blood and tumor samples, with distinct profiles in the tumor compared to peripheral blood samples (FIGS. 5, 13, 14). Intratumoral B-cells had reduced expression of CD21, CD23, CD79b, and CXCR5, pointing to distinct functional and migratory profiles compared to similar B-cell populations in the peripheral blood (FIG. 15A). The phenotypes of B-cells in Rs as compared to NRs to ICB were next compared in both tumor and peripheral blood. Though B-cell subsets (naïve, memory and transitional B-cells and plasma cells) in the peripheral blood had a similar distribution in R and NR (FIG. 13B), significant differences were noted in B-cell subsets in tumors of R vs NR to ICB (FIG. 13B). Specifically, tumors from R had a significantly higher frequency of memory B-cells, whereas NR had a significantly higher frequency of naïve B-cells (p=0.033 for naïve and p=0.033 for memory) (FIGS. 5, 13). Other notable differences included an increase in plasma cells in R vs NR; however, this did not reach significance and was largely driven by data from one patient (p=0.3). An increased percentage of CXCR3⁺ and CD86⁺ B-cells were identified in Rs as compared to NRs, which are markers of memory B-cells and germinal center B-cells (FIG. 15).

In summary, multiomic data is presented supporting a role for B-cells within TLS in the response to ICB in patients with melanoma and RCC. While the distinct mechanisms through which B-cells contribute are incompletely understood, the data suggests that the same properties of memory B-cells and plasma cells desirable for acquired immune responses may also be contributing to an effective T-cell response following ICB. Importantly, these B-cells are likely acting in concert with other key immune constituents of the TLS by altering T-cell activation and function as well as through other mechanisms. Memory B-cells may be acting as antigen-presenting cells, driving the expansion of both memory and naïve tumor-associated T-cell responses. B-cells can also secrete an array of cytokines, including TNF-α, IL-2, IL-6 and IFNγ, through which they activate and recruit other immune effector cells, including T-cells. The observation of switched memory B-cells (that can differentiate into plasma cells) in responders suggests that they could be potentially contributing to the anti-tumor response by producing antibodies against tumor antigens. This represents an important insight into therapeutic responses to ICB and will likely stimulate further research in this area.

TABLE 1
Cohort Characteristics of high risk resectable melanoma patients treated with neoadjuvant immune checkpoint blockade.

| | | | | | Stage | | | Tumor | | | |
|---|---|---|---|---|---|---|---|---|---|---|---|
| | | | | | AJCC | | Primary | PD- | | | |
| Patient | Tx | RECIST | Age | | 7th | LDH > | tumor | L1 ≥ | BRAF | Disease | Previous |
| ID | Type | Response* | (yrs) | Gender | Edition | ULN | type | 1% | mutation? | Site | Therapies |
| 1 | I + N | R | 48 | M | IIIB | No | Superficial | Yes | Yes | Lymph | Surgical |
| | | | | | | | spreading | | (V600E) | node | resection |
| 3 | I + N | NR | 29 | M | IV M1a | No | Superficial | No | Yes | Lymph | Surgical |
| | | | | | | | spreading | | (V600E) | node | resection |
| | | | | | | | | | | | 6 weeks |
| | | | | | | | | | | | interferon |
| 6 | I + N | R | 74 | M | IIIC | No | Unclassified | Yes | No | Subcut. | None |
| | | | | | | | type | | | nodule | |
| 8 | I + N | NR | 38 | F | IIIC | No | Unknown | No | Yes | Lymph | Surgical |
| | | | | | | | primary | | (V600E) | node | resection |
| 10 | I + N | R | 49 | M | IIIC | No | Nodular | Yes | Yes | Lymph | Surgical |
| | | | | | | | | | (V600E) | node | resection |
| 13 | I + N | R | 44 | M | IIIC | No | Unknown | Yes | No | Lymph | Surgical |
| | | | | | | | primary | | | node | resection |
| 14 | I + N | NR | 49 | M | IIIC | No | Nodular | No | No | Subcut. | Surgical |
| | | | | | | | | | | nodule | resection, |
| | | | | | | | | | | | interferon |
| 15 | I + N | R | 66 | M | IV M1a | No | Unknown | Yes | No | Lymph | None |
| | | | | | | | primary | | | node | |
| 17 | I + N | R | 45 | M | IIIB | No | Nodular | Yes | No | Subcut. | None |
| | | | | | | | | | | nodule | |
| 19 | I + N | R | 70 | M | IV M1a | No | Nodular | Yes | No | Lymph | Surgical |
| | | | | | | | | | | node | resection, |
| | | | | | | | | | | | peg-interferon |
| 20 | I + N | R | 58 | M | IIIB | No | Unknown | No | No | Lymph | None |
| | | | | | | | primary | | | node | |
| 2 | N | R | 67 | F | IIIB | No | Superficial | Yes | No | Lymph | Surgical |
| | | | | | | | spreading | | | node | resection |
| 4 | N | R | 56 | M | IIIB | No | Unknown | No | Yes | Lymph | None |
| | | | | | | | primary | | (V600E) | node | |
| 5 | N | NR | 63 | M | IIIB | Yes | Superficial | No | Yes | Lymph | None |
| | | | | | | | spreading | | (V600E) | node | |
| 7 | N | NR | 34 | M | IIIB | No | Unclassified | Yes | No | Lymph | None |
| | | | | | | | type | | | node | |
| 9 | N | NR | 40 | M | IIIC | No | Superficial | Yes | Yes | Lymph | Surgical |
| | | | | | | | spreading | | (V600E) | node | resection |
| 11 | N | NR | 64 | M | IV M1c | Yes | Unknown | Yes | Yes | Lymph | None |
| | | | | | | | primary | | (V600E) | node | |
| 12 | N | NR | 46 | M | IIIC | No | Nodular | Yes | Yes | Subcut. | Surgical |
| | | | | | | | | | (V600E) | nodule | resection, |
| | | | | | | | | | | | radiation |
| | | | | | | | | | | | therapy, |
| | | | | | | | | | | | IL-2, interferon, |
| | | | | | | | | | | | dabrafenib + |
| | | | | | | | | | | | trametinib |
| 16 | N | NR | 48 | F | IIIB | No | Superficial | No | Yes | Lymph | Surgical |
| | | | | | | | spreading | | (V600E) | node | resection |
| 18 | N | NR | 60 | M | IIIC | No | Superficial | Yes | Yes | Subcut. | None |
| | | | | | | | spreading | | (V600R) | nodule | |
| 21 | N | NR | 54 | F | IIIC | No | Unclassified | Yes | No | Subcut. | None |
| | | | | | | | type | | | nodule | |
| 22 | N | NR | 47 | M | IIIB | No | Unknown | No | No | Lymph | None |
| | | | | | | | primary | | | node | |
| 23 | N | R | 73 | M | IIIC | No | Lentigo | Yes | No | Lymph | Surgical |
| | | | | | | | maligna | | | node | resection |

Tx, treatment; I + N, ipilimumab with nivolumab; N, nivolumab; R, responder; NR, non-responder; LDH, lactate dehydrogenase; subcut., subcutaneous
*Response defined as achieving a complete or partial response by RECIST 1.1.

TABLE 2
Cohort characteristics of renal cell carcinoma (RCC) patients.

| | | Response per | Age | | | |
|---|---|---|---|---|---|---|
| Patient ID | Tx Type | RECIST* | (yrs) | Gender | Disease Site | Previous Treatment |
| 1444 | I + N | PR | 57 | M | Liver | Radical nephrectomy |
| 1578 | I + N | PD | 47 | M | Retroperitoneal LN | Radical nephrectomy |
| 1729 | N | PD | 69 | M | Adrenal gland | Radical adrenal-sparing |
| | | | | | | nephrectomy |
| 761 | N + Bev | PD | 44 | F | Liver | Nephrectomy |
| 774 | N | PR | 67 | M | Kidney | No |
| 1884 | N | PR | 60 | F | Pelvic LN | Adrenal-sparing radical |
| | | | | | | nephrectomy |
| 1326 | I + N | PR | 67 | M | Lung | Radical nephrectomy, |
| | | | | | | adrenalectomy |
| 1551 | N + Bev | PR | 56 | F | Kidney | Radical nephrectomy |
| 1442 | N + Bev | PD | 46 | M | Liver | None |
| 1446 | I + N | PR | 46 | M | Kidney | None |
| 1605 | N + Bev | PD* | 52 | F | Lung | Cytoreductive nephrectomy, |
| | | | | | | palliative XRT hip |
| 1620 | N + Bev | PR | 68 | M | Kidney | None |
| 1678 | N + Bev | PR | 66 | M | Kidney | None |
| 1698 | N + Bev | PR | 47 | M | Pelvic Peritoneal | Nephrectomy |
| 1788 | N + Bev | PR | 48 | M | Pleura | Nephrectomy |
| 1802 | N + Bev | PD | 67 | M | Retroperitoneal | Sutent/Votrient |
| 2112 | I + N | PD | 47 | M | Rib | Radical nephrectomy |
| 1971 | N + Bev | PR | 65 | M | Retroperitoneal LN | Radical nephrectomy |
| 1859 | N + Bev | PD | 52 | M | Lung | Radical nephrectomy |
| 1989 | N | PR | 52 | M | Adrenal gland | Radical nephrectomy |
| 1906 | I + N | PD | 64 | M | Retroperitoneal LN | Radical nephrectomy |
| 1881 | N | PR | 62 | M | Kidney | None |
| 1933 | N + Bev | PR | 63 | F | Kidney | None |
| 1974 | N + Bev | PR | 71 | M | Kidney | None |
| 1983 | N | PR | 57 | M | Spine | None |
| 2056 | I + N | PD | 63 | M | Kidney | None |
| 2064 | N + Bev | PR | 68 | M | Kidney | Partial nephrectomy, |
| | | | | | | radiofrequency ablation |
| 1889 | I + N | PD | 69 | M | Kidney | Cryoablation of clavicle |

Tx, treatment; I + N, ipilimumab with nivolumab; N, nivolumab; N + Bev, nivolumab with bevacizumab; R, responder; NR, non-responder; LN, lymph node
*Clinical response is defined as achieving a partial response (PR) or complete response (CR) at 12 weeks by RECIST 1.1.
#This patient has PD as defined by primary endpoint of the clinical trial, but a R per best overall response.

TABLE 3
Unvariable and multivariable logistic regression analysis
predicting response to melanoma neoadjuvant ICB using
immune cell signatures at baseline and on-treatment.

| | Univariable (n = 41) | | Multivariable (n = 41) | |
|---|---|---|---|---|
| Cell Lineage | OR | p-value | OR | p-value |
| T cells | 2.6 | 0.02 | 0.8 | 0.9 |
| CD8 T cells | 2.6 | 0.02 | 0.4 | 0.5 |
| Cytotoxic lymphocytes | 3.3 | 0.01 | 3.6 | 0.3 |
| NK cells | 3.1 | 0.01 | 1.3 | 0.8 |
| B lineage | 2.6 | 0.02 | 2.1 | 0.4 |
| Monocytic lineage | 2.4 | 0.03 | 1.3 | 0.7 |
| Myeloid dendritic cells | 1.5 | 0.2 | | |
| Neutrophils | 0.8 | 0.6 | | |
| Endothelial cells | 1.0 | 0.9 | | |

For Patients Treated with Nivolumab Only

Univariable (n = 22)

| Cell Lineage | OR | p-value |
|---|---|---|
| T cells | 3.8 | 0.06 |
| CD8 T cells | 2.8 | 0.1 |
| Cytotoxic lymphocytes | 2.6 | 0.07 |
| NK cells | 3.0 | 0.08 |
| B lineage | 4.2 | 0.04 |
| Monocytic lineage | 2.8 | 0.1 |
| Myeloid dendritic cells | 2.1 | 0.2 |
| Neutrophils | 0.7 | 0.5 |
| Endothelial cells | 1.5 | 0.5 |

For Patients treated with Ipilimumab + Nivolumab

Univariable (n = 19)

| Cells | OR | p-value |
|---|---|---|
| T cells | 1.7 | 0.4 |
| CD8 T cells | 1.9 | 0.2 |
| Cytotoxic lymphocytes | 4.3 | 0.09 |
| NK cells | 3.3 | 0.07 |
| B lineage | 1.5 | 0.5 |
| Monocytic lineage | 2.8 | 0.1 |
| Myeloid dendritic cells | 1.1 | 0.9 |
| Neutrophils | 0.8 | 0.7 |
| Endothelial cells | 1.04 | 0.9 |

ICB, immune checkpoint blockade;
OR, odds ratio

TABLE 4
Univariable and multivariable logistic regression analysis
predicting response to melanoma for neoadjuvant ICB (two
patient cohorts) using immune cell signatures.

| | Univariable (n = 39) | | Multivariable (n = 39) | |
|---|---|---|---|---|
| Cell Lineage | OR | p-value | OR | p-value |
| T cells | 3.6 | 0.01 | 0.97 | 0.98 |
| CD8 T cells | 3.6 | 0.01 | 3.4 | 0.4 |
| Cytotaxic lymphocytes | 2.2 | 0.04 | 0.7 | 0.6 |
| NK cells | 1.9 | 0.1 | | |
| B lineage | 3.0 | 0.03 | 1.9 | 0.3 |
| Monocytic lineage | 2.5 | 0.03 | 1.7 | 0.4 |
| Myeloid dendritic cells | 2.4 | 0.05 | 0.7 | 0.6 |
| Neutrophils | 1.2 | 0.6 | | |
| Endothelial cells | 0.7 | 0.4 | | |

ICB, in.Tirritine checkpoint blockade;
OR, odds ratio

TABLE 5
Univariable and multivariable logistic regression analysis
predicting response to pre-surgical ICB in RCC patients using
immune cell signatures.
Extended Data Table 6. Univariable and multivariable logistic
regression analysis predicting response to pre-surgical
ICB in RCC patients using immune cell signatures.

| | Univariable (n = 28) | | Multivariable (n = 28) | |
|---|---|---|---|---|
| Cell Lineage | OR | p-value | OR | p-value |
| T cells | 28.2 | 0.01 | 10.6 | 0.2 |
| CD8 T cells | 432.6 | 0.02 | 121.5 | 0.2 |
| Cytotoxic lymphocytes | 6.7 | 0.02 | 0.92 | 0.9 |
| NK cells | 1.8 | 0.1 | | |
| B lineage | 61.2 | 0.05 | 2.0 | 0.6 |
| Monocytic lineage | 2.4 | 0.09 | | |
| Myeloid dendritic cells | 2.4 | 0.1 | | |
| Neutrophils | 1.4 | 0.3 | | |
| Endothelial cells | 1.3 | 0.4 | | |

For Patients Treated with Nivolumab Only

Univariable (n = 6)

| | | |
|---|---|---|
| Cell Lineage | OR | p-value |

| | |
|---|---|
| T cells | Unable to evaluate due to only 1 patient |
| CD8 T cells | in NR category |
| Cytotoxic lymphocytes | |
| NK cells | |
| B lineage | |
| Monocytic lineage | |
| Myeloid dendritic cells | |
| Neutrophils | |
| Endothelial cells | |

For Patients treated with Ipilimumab + Nivolumab
or Nivolumab + Bevacizumab

Univariable (n = 22)

| Cells | OR | p-value |
|---|---|---|
| T cells | 17.0 | 0.02 |
| CD8 T cells | 683.3 | 0.05 |
| Cytotoxic lymphocytes | 5.4 | 0.04 |
| NK cells | 1.8 | 0.2 |
| B lineage | 128.0 | 0.06 |
| Monocytic lineage | 2.5 | 0.07 |
| Myeloid dendritic cells | 2.2 | 0.1 |
| Neutrophils | 1.5 | 0.4 |
| Endothelial cells | 1.6 | 0.2 |

ICB, immune checkpoint blockade;
RCC, renal cell carcinoma;
OR, odds ratio

TABLE 6
Summary of univariable and multivariable Cox Proportional
Hazard models testing prognostic value of immune
signatures calculated using TCGA SKCM data.

| | | Univariable | | Multivariable | |
|---|---|---|---|---|---|
| | | (n = 27) | | (n = 127) | |
| Cell Lineage | High versus Low | HR | p-value | HR | p-value |
| T cells | High | 1.0 | — | 1.0 | — |
| | Low | 1.9 | 0.03 | 1.3 | 0.7 |
| CDS T cells | High | 1.0 | — | 1.0 | — |
| | Low | 1.7 | 0.05 | 0.7 | 0.6 |
| Cytotoxic lymphocytes | High | 1.0 | — | | |
| | Low | 1.5 | 0.1 | | |
| NK cells | High | 1.0 | — | 1.0 | — |
| | Low | 1.8 | 0.04 | 1.5 | 0.4 |
| B lineage | High | 1.0 | — | 1.0 | — |
| | Low | 1.7 | 0.05 | 1.3 | 0.5 |
| Monocytic lineage | High | 1.0 | — | 1,0 | — |
| | Low | 1.9 | 0.02 | 1.5 | 0.3 |
| Myeloid dendritic cells | High | 1.0 | — | | |
| | Low | 1.5 | 0.1 | | |
| Neutrophils | High | 1.0 | — | | |
| | Low | 1.1 | 0.6 | | |
| Endothelial cells | High | 1.0 | — | | |
| | Low | 0.8 | 0.5 | | |

HR is the Hazard Ratio, the median values were used for dichotomizing cell types into high and low

TABLE 7
Pathways associated with overexpressed genes at baseline in Responder and Non-responder
patients treated with neoadjuvant immune checkpoint blockade as analyzed by bulk RNAseq.

| | | |
|---|---|---|
| Pathway | P | FDR |

Pathways associated with overexpressed genes in Responders pre-treatment

| | | |
|---|---|---|
| IL12-mediated signaling events(N) | 2.65E−13 | 3.97E−11 |
| Downstream signaling in naive CD8+ T cells(N) | 4.05E−13 | 3.97E−11 |
| Antigen processing and presentation(K) | 1.00E−10 | 6.53E−09 |
| Primary immunodeficiency(K) | 1.96E−09 | 9.59E−08 |
| IL12 signaling mediated by STAT4(N) | 4.95E−08 | 1.93E−06 |
| Th1 and Th2 cell differentiation(K) | 4.09E−07 | 1.31E−05 |
| Hematopoietic cell lineage(K) | 5.56E−07 | 1.56E−05 |
| TCR signaling in naive CD8+ T cells(N) | 7.54E−07 | 1.81E−05 |
| Th17 cell differentiation(K) | 9.79E−07 | 2.06E−05 |
| Class I MHC mediated antigen processing & presentation(R) | 1.87E−06 | 3.49E−05 |
| lmmunoregulatory interactions between a Lymphoid and a non- | 2.07E−06 | 3.49E−05 |
| Lymphoid cell(R) | | |
| TCR signaling(R) | 2.18E−06 | 3.49E−05 |
| Measles(K) | 3.87E−06 | 5.81E−05 |
| Ick and fyn tyrosine kinases in initiation of tcr activation(B) | 6.08E−06 | 8.51E−05 |
| Graft-versus-host disease(K) | 8.63E−06 | 1.12E−04 |
| Proteasome(K) | 1.14E−05 | 1.36E−04 |
| Cadherin signaling pathway(P) | 1.48E−05 | 1.57E−04 |
| Cytokine-cytokine receptor interaction(K) | 1.57E−05 | 1.57E−04 |
| T cell receptor signaling pathway(K) | 1.87E−05 | 1.87E−04 |
| IL2-mediated signaling events(N) | 2.52E−05 | 2.27E−04 |
| the co-stimulatory signal during t-cell activation(B) | 3.58E−05 | 3.22E−04 |
| Costimulation by the CD28 family(R) | 5.17E−05 | 4.13E−04 |
| role of mef2d in t-cell apoptosis(B) | 5.41E−05 | 4.33E−04 |
| Natural killer cell mediated cytotoxicity(K) | 6.14E−05 | 4.85E−04 |
| ras-independent pathway in nk cell-mediated cytotoxicity(B) | 6.92E−05 | 4.85E−04 |
| T cell activation(P) | 1.20E−04 | 8.42E−04 |
| CXCR3-mediated signaling events(N) | 1.86E−04 | 1.30E−03 |
| IL23-mediated signaling events(N) | 2.19E−04 | 1.53E−03 |
| TNFR2 non-canonical NF-kB pathway(R) | 2.78E−04 | 1.67E−03 |
| activation of csk by camp-dependent protein kinase inhibits signaling | 2.95E−04 | 1.77E−03 |
| through the t cell receptor(B) | | |
| Chagas disease (American trypanosomiasis)(K) | 3.11E−04 | 1.86E−03 |
| IL2 receptor beta chain in t cell activation(B) | 3.62E−04 | 1.94E−03 |
| RAF/MAP kinase cascade(R) | 3.88E−04 | 1.94E−03 |
| t cell receptor signaling pathway(B) | 4.67E−04 | 2.33E−03 |
| SHP2 signaling(N) | 5.56E−04 | 2.78E−03 |
| IL2 signaling pathway(B) | 5.63E−04 | 2.81E−03 |
| Regulation of Apoptosis(R) | 6.21E−04 | 3.11E−03 |
| Hedgehog ligand biogenesis(R) | 1.02E−03 | 4.87E−03 |
| HTLV-I infection(K) | 1.19E−03 | 4.87E−03 |
| TCR signaling in naive CD4+ T cells(N) | 1.22E−03 | 4.87E−03 |
| Metabolism of polyamines(R) | 1.22E−03 | 4.87E−03 |
| Degradation of beta-catenin by the destruction complex(R) | 1.38E−03 | 5.52E−03 |
| Wnt signaling pathway(P) | 1.38E−03 | 5.53E−03 |
| Regulation of Hypoxia-inducible Factor (HIF) by oxygen(R) | 1.56E−03 | 6.22E−03 |
| Interferon gamma signaling(R) | 1.56E−03 | 6.22E−03 |
| Regulation of DNA replication(R) | 1.62E−03 | 6.47E−03 |
| CXCR4-mediated signaling events(N) | 2.02E−03 | 7.18E−03 |
| Hedgehog ‘on’ state(R) | 2.16E−03 | 7.18E−03 |
| Regulation of mitotic cell cycle(R) | 2.24E−03 | 7.18E−03 |
| M/G1 Transition(R) | 2.39E−03 | 7.18E−03 |
| Regulation of mRNA stability by proteins that bind AU-rich elements(R) | 2.56E−03 | 7.67E−03 |
| Herpes simplex infection(K) | 2.60E−03 | 7.80E−03 |
| Chemokine signaling pathway(K) | 2.70E−03 | 8.11E−03 |
| ABC-family proteins mediated transport(R) | 2.72E−03 | 8.17E−03 |
| MAPK6/MAPK4 signaling(R) | 2.72E−03 | 8.17E−03 |
| Hedgehog ‘off’ state(R) | 2.81E−03 | 8.43E−03 |
| IL2 signaling events mediated by STAT5(N) | 3.53E−03 | 0.0106 |
| Synthesis of DNA(R) | 3.57E−03 | 0.0107 |
| Toll-like receptor signaling pathway(K) | 4.44E−03 | 0.0127 |
| G-protein beta:gamma signalling(R) | 4.57E−03 | 0.0127 |
| Beta-catenin independent WNT signaling(R) | 5.17E−03 | 0.0127 |
| African trypanosomiasis(K) | 5.44E−03 | 0.0127 |
| IL2 signaling events mediated by PI3K(N) | 5.44E−03 | 0.0127 |
| Deubiquitination(R) | 5.54E−03 | 0.0127 |
| Allograft rejection(K) | 6.37E−03 | 0.0127 |
| Interleukin-3, 5 and GM-CSF signaling(R) | 6.37E−03 | 0.0127 |
| IFN-gamma pathway(N) | 7.03E−03 | 0.0141 |
| Interleukin-2 family signaling(R) | 7.03E−03 | 0.0141 |
| Type I diabetes mellitus(K) | 8.08E−03 | 0.0162 |
| Mitotic G1-G1/S phases(R) | 8.11E−03 | 0.0162 |
| DAP12 interactions(R) | 8.44E−03 | 0.0169 |
| S Phase(R) | 8.45E−03 | 0.0169 |
| ABC transporters(K) | 8.82E−03 | 0.0176 |
| Interleukin-1 family signaling(R) | 8.97E−03 | 0.0179 |
| C-type lectin receptors (CLRs)(R) | 9.33E−03 | 0.0187 |
| Apoptosis(K) | 9.52E−03 | 0.019 |
| Interleukin-10 signaling(R) | 9.58E−03 | 0.0192 |
| Toll-Like Receptors Cascades(R) | 0.0101 | 0.0202 |
| GPVI-mediated activation cascade(R) | 0.0104 | 0.0207 |
| Cell adhesion molecules (CAMs)(K) | 0.0105 | 0.0209 |
| Caspase cascade in apoptosis(N) | 0.0116 | 0.0232 |
| Phagosome(K) | 0.0123 | 0.0246 |
| Jak-STAT signaling pathway(K) | 0.0132 | 0.0263 |
| TCF dependent signaling in response to WNT(R) | 0.0136 | 0.0272 |
| Necroptosis(K) | 0.0141 | 0.0278 |
| Class A/1 (Rhodopsin-like receptors)(R) | 0.0152 | 0.0278 |
| Mitotic Metaphase and Anaphase(R) | 0.0162 | 0.0278 |
| Influenza A(K) | 0.0173 | 0.0278 |
| Signaling by the B Cell Receptor (BCR)(R) | 0.0175 | 0.0278 |
| Inflammatory bowel disease (IBD)(K) | 0.0177 | 0.0278 |
| Mitotic G2-G2/M phases(R) | 0.018 | 0.0278 |
| Transcriptional misregulation in cancer(K) | 0.0186 | 0.0278 |
| Regulation of Telomerase(N) | 0.0192 | 0.0278 |
| Interferon alpha/beta signaling(R) | 0.0198 | 0.0278 |
| G alpha (i) signalling events(R) | 0.0221 | 0.0278 |
| Signaling by ROBO receptors(R) | 0.0247 | 0.0278 |
| Glucocorticoid receptor regulatory network(N) | 0.0248 | 0.0278 |
| Fc epsilon receptor (FCERI) signaling(R) | 0.0274 | 0.0278 |
| sodd/tnfr1 signaling pathway(B) | 0.0278 | 0.0278 |
| PIP3 activates AKT signaling(R) | 0.0319 | 0.0319 |
| Neddylation(R) | 0.0323 | 0.0323 |
| il-7 signal transduction(B) | 0.0338 | 0.0338 |
| Histidine, lysine, phenylalanine, tyrosine, proline and tryptophan | 0.0338 | 0.0338 |
| catabolism(R) | | |
| IL-17 signaling pathway(K) | 0.0343 | 0.0343 |
| role of mitochondria in apoptotic signaling(B) | 0.0399 | 0.0399 |
| Cell Cycle Checkpoints(R) | 0.0413 | 0.0413 |
| il 4 signaling pathway(B) | 0.0429 | 0.0429 |
| Interferon-gamma signaling pathway(P) | 0.0459 | 0.0459 |
| Regulated Necrosis(R) | 0.0459 | 0.0459 |
| TNF signaling pathway(K) | 0.0464 | 0.0464 |
| tnfr1 signaling pathway(B) | 0.0518 | 0.0518 |
| Toxoplasmosis(K) | 0.0526 | 0.0526 |
| Clathrin-mediated endocytosis(R) | 0.0526 | 0.0526 |

Pathways associated with overexpressed genes in Non-responders pre-treatment

| | | |
|---|---|---|
| PI3K-Akt signaling pathway(K) | 4.18E−05 | 6.52E−03 |
| FGF signaling pathway(P) | 1.89E−04 | 0.0106 |
| Extracellular matrix organization(R) | 2.46E−04 | 0.0106 |
| Chagas disease (American trypanosomiasis)(K) | 2.71E−04 | 0.0106 |
| Glypican 1 network(N) | 4.46E−04 | 0.0138 |
| Hepatitis C(K) | 5.54E−04 | 0.0144 |
| Phospholipase D signaling pathway(K) | 6.98E−04 | 0.0154 |
| Netrin-1 signaling(R) | 1.69E−03 | 0.0305 |
| Proteoglycans in cancer(K) | 1.93E−03 | 0.0305 |
| Rap1 signaling pathway(K) | 2.12E−03 | 0.0305 |
| Alzheimer disease-amyloid secretase pathway(P) | 2.43E−03 | 0.0305 |
| Shigellosis(K) | 2.93E−03 | 0.0305 |
| Beta1 integrin cell surface interactions(N) | 3.02E−03 | 0.0305 |
| Plasma lipoprotein assembly, remodeling, and clearance(R) | 3.20E−03 | 0.0305 |
| Epithelial cell signaling in Helicobacter pylori infection(K) | 3.20E−03 | 0.0305 |
| RIG-I-like receptor signaling pathway(K) | 3.39E−03 | 0.0305 |
| Pertussis(K) | 3.98E−03 | 0.0354 |
| L1CAM interactions(R) | 4.61E−03 | 0.0354 |
| ECM-receptor interaction(K) | 4.61E−03 | 0.0354 |
| Salmonella infection(K) | 5.06E−03 | 0.0354 |
| IL-17 signaling pathway(K) | 5.88E−03 | 0.0412 |
| Huma papillomavirus infection(K) | 6.32E−03 | 0.0414 |
| AGE-RAGE signaling pathway in diabetic complications(K) | 6.90E−03 | 0.0414 |
| Retrograde endocannabinoid signaling(K) | 6.90E−03 | 0.0414 |
| Toll-like receptor signaling pathway(K) | 7.57E−03 | 0.0454 |
| Sphingolipid signaling pathway(K) | 9.61E−03 | 0.053 |
| Dopaminergic synapse(K) | 0.0112 | 0.053 |
| Fox() signaling pathway(K) | 0.0119 | 0.053 |
| Pathways in cancer(K) | 0.0121 | 0.053 |
| regulators of bone mineralization(B) | 0.0124 | 0.053 |
| Fluid shear stress and atherosclerosis(K) | 0.0127 | 0.053 |
| Signaling pathways regulating pluripotency of stem cells(K) | 0.0132 | 0.053 |
| Signaling mediated by p38-gamma and p38-delta(N) | 0.0137 | 0.0547 |

FDR refers to the False Discovery Rate;
Biological databases used (K- KEGG data, R- Reactome Pathway Database, N- NCBI database, P- Panther Database, and B- Biocarta database)

TABLE 8
Pathways associated with overexpressed genes in Responders and
Nonresponders on-treatment in melanoma patients treated with neoadjuvant
immune checkpoint blockade as analyzed by bulk RNAseq.

| | | |
|---|---|---|
| Pathway | p-Value | FDR |

Pathways associated with overexpressed genes in Responders on-treatment

| | | |
|---|---|---|
| Extracellular matrix organization(R) | 8.40E−06 | 1.50E−03 |
| Graft-versus-host disease(K) | 6.98E−05 | 6.21E−03 |
| Validated targets of C-MYC transcriptional repression(N) | 2.03E−04 | 6.45E−03 |
| Cytokine-cytokine receptor interaction(K) | 2.16E−04 | 6.45E−03 |
| IL12-mediated signaling events(N) | 2.24E−04 | 6.45E−03 |
| granzyme a mediated apoptosis pathway(B) | 2.57E−04 | 6.45E−03 |
| Downstream signaling in naive CD8+ T cells(N) | 2.58E−04 | 6.45E−03 |
| Antigen processing and presentation(K) | 4.42E−04 | 8.53E−03 |
| Chemokine signaling pathway(K) | 4.49E−04 | 8.53E−03 |
| Rheumatoid arthritis(K) | 6.94E−04 | 0.0118 |
| Hematopoietic cell lineage(K) | 8.61E−04 | 0.0138 |
| ras-independent pathway in nk cell-mediated cytotoxicity(B) | 1.10E−03 | 0.0154 |
| Leukocyte transendothelial migration(K) | 1.30E−03 | 0.0165 |
| IL2 signaling events mediated by STAT5(N) | 1.37E−03 | 0.0165 |
| Syndecan-4-mediated signaling events(N) | 1.78E−03 | 0.0196 |
| Natural killer cell mediated cytotoxicity(K) | 2.03E−03 | 0.022 |
| Validated transcriptional targets of AP1 family members Fra1 and | 2.37E−03 | 0.022 |
| Fra2(N) | | |
| Allograft rejection(K) | 2.50E−03 | 0.022 |
| Cell adhesion molecules (CAMs)(K) | 2.66E−03 | 0.022 |
| Class A/1 (Rhodopsin-like receptors)(R) | 2.75E−03 | 0.022 |
| Pathways in cancer(K) | 2.95E−03 | 0.0233 |
| Type I diabetes mellitus(K) | 3.18E−03 | 0.0233 |
| DAP12 interactions(R) | 3.32E−03 | 0.0233 |
| Intestinal immune network for IgA production(K) | 4.10E−03 | 0.0287 |
| Autoimmune thyroid disease(K) | 4.77E−03 | 0.0288 |
| G alpha (i) signalling events(R) | 4.80E−03 | 0.0288 |
| TCR signaling in naive CD8+ T cells(N) | 4.95E−03 | 0.0297 |
| Viral myocarditis(K) | 5.87E−03 | 0.0352 |
| Proteoglycans in cancer(K) | 6.72E−03 | 0.0386 |
| Epithelial cell signaling in Helicobacter pylori infection(K) | 7.72E−03 | 0.0386 |
| inhibition of matrix metalloproteinases(B) | 7.76E−03 | 0.0388 |
| EGF receptor signaling pathway(P) | 0.011 | 0.0552 |

Pathways associated with overexpressed genes in Non-responders on-treatment

| | | |
|---|---|---|
| Olfactory transduction(K) | 1.11E−16 | 6.43E−14 |
| G alpha (s) signalling events(R) | 7.45E−14 | 2.15E−11 |
| PI3K-Akt signaling pathway(K) | 3.21E−08 | 6.20E−06 |
| ECM-receptor interaction(K) | 2.87E−06 | 4.13E−04 |
| Focal adhesion(K) | 1.20E−05 | 1.38E−03 |
| Arrhythmogenic right ventricular cardiomyopathy (ARVC)(K) | 3.96E−05 | 3.81E−03 |
| Cholinergic synapse(K) | 4.97E−05 | 4.07E−03 |
| Neurotransmitter receptors and postsynaptic signal transmission(R) | 1.09E−04 | 7.83E−03 |
| Autoimmune thyroid disease(K) | 2.06E−04 | 0.012 |
| Dilated cardiomyopathy (DCM)(K) | 2.10E−04 | 0.012 |
| Human papillomavirus infection(K) | 2.32E−04 | 0.012 |
| Ion channel transport(R) | 4.78E−04 | 0.0226 |
| Chemical carcinogenesis(K) | 5.51E−04 | 0.0226 |
| Cytosolic DNA-sensing pathway(K) | 5.73E−04 | 0.0226 |
| Hypertrophic cardiomyopathy (HCM)(K) | 5.95E−04 | 0.0226 |
| Pathways in cancer(K) | 7.16E−04 | 0.0258 |
| Extracellular matrix organization(R) | 8.48E−04 | 0.0288 |
| Glutamatergic synapse(K) | 1.12E−03 | 0.0336 |
| Plasminogen activating cascade(P) | 1.12E−03 | 0.0336 |
| Circadian entrainment(K) | 1.50E−03 | 0.0405 |
| Dissolution of Fibrin Clot(R) | 1.51E−03 | 0.0405 |
| Metabolism of xenobiotics by cytochrome P450(K) | 1.56E−03 | 0.0405 |
| Cadherin signaling pathway(P) | 1.92E−03 | 0.0481 |
| Oxytocin signaling pathway(K) | 2.31E−03 | 0.0547 |
| Long-term depression(K) | 2.38E−03 | 0.0547 |

FDR refers to the False Discovery Rate;
Biological databases used (K- KEGG data, R- Reactome Pathway Database, N- NCBI database, P- Panther Database, and B- Biocarta database)

TABLE 9
Summary of patient samples included in the analyses performed on patients with high-risk resectable melanoma treated with neoadjuvant ICB.

| | Bulk tumor | | | | | | | | | | Analysis | | |
|---|---|---|---|---|---|---|---|---|---|---|---|---|---|
| | transcriptional | | | | | | | | BCR | | of | | |
| | analyses | | | | | Single Cell | | | analysis | | Serum | | |
| | MCP | | | | | RNA- | | Mass | (by bulk | | Exosomes | | Nano- |
| Patient | Counter | | For | IHC | | sequencing | | Cyto- | RNAseq) | | | On- | string |
| ID | B | On-Tx | DGE | B | On-Tx | B | On-Tx | metry | B | On-Tx | B | Tx | DSP |
| 1 | x | x | x | x | x | x | x | Refer to | x | x | NA | x | x |
| 2 | x | x | x | x | x | NA | x | the | x | x | x | x | x |
| 3 | x | x | NA | x | x | NA | NA | Extended | x | x | x | x | NA |
| 4 | x | x | x | x | x | NA | NA | Data Table | x | x | x | x | NA |
| 5 | x | x | NA | x | x | NA | NA | 15 | x | x | NA | NA | NA |
| 6 | x | x | x | NA | NA | NA | NA | | x | x | NA | NA | NA |
| 7 | x | x | x | x | NA | NA | NA | | x | x | NA | NA | NA |
| 8 | x | x | x | x | x | NA | NA | | x | x | x | x | NA |
| 9 | x | x | x | x | x | NA | NA | | x | x | x | x | NA |
| 10 | x | x | NA | x | x | NA | NA | | x | x | x | x | x |
| 11 | x | x | x | x | x | NA | NA | | x | x | NA | NA | NA |
| 12 | x | x | x | x | x | NA | NA | | x | x | NA | NA | NA |
| 13 | x | x | x | x | x | NA | NA | | x | x | x | x | NA |
| 14 | x | x | x | x | x | NA | NA | | x | x | NA | NA | NA |
| 15 | x | x | x | x | x | NA | NA | | x | x | NA | NA | NA |
| 16 | x | x | x | x | x | NA | NA | | x | x | x | x | NA |
| 17 | NA | NA | NA | x | x | NA | NA | | NA | NA | x | x | x |
| 18 | x | NA | x | x | x | NA | NA | | x | NA | x | x | NA |
| 19 | x | NA | NA | x | x | NA | NA | | x | NA | x | x | x |
| 20 | x | x | x | x | NA | NA | NA | | x | x | NA | NA | NA |
| 21 | x | x | x | NA | x | NA | NA | | x | x | NA | NA | NA |
| 22 | NA | x | NA | x | x | NA | NA | | NA | x | NA | NA | NA |
| 23 | x | x | NA | x | x | NA | NA | | x | x | x | x | NA |

NA-sample not available; B refers to the Baseline; On-Tx refers to the on-treatment; BCR is B-cell Receptor; IHC is immunohistochemistry; DGE-differential gene expression analysis

TABLE 10
Summary of patient samples included in mass cytometry analyses.

| Patient ID | Tx Type | REC1ST Response+ | Disease Site | PBMCs Timepoint | Tumor Timepoint |
|---|---|---|---|---|---|
| 2 | N | R | Lymph node | On Treatment* | Surgery* |
| 4 | N | R | Lymph node | X | X |
| 23 | N | R | Lymph node | X | On Treatment |
| 1 | I + N | R | Lymph node | X | Surgery |
| 6 | I + N | R | Subcut. nodule | X | insufficient |
| 10 | I + N | R | Lymph node | X | insufficient |
| 13 | I + N | R | Lymph node | On Treatment* | Surgery* |
| 15 | I + N | R | Lymph node | On Treatment* | On Treatment* |
| 17 | I + N | R | Subcut. nodule | On Treatment* | On Treatment* |
| 19 | I + N | R | Lymph node | X | Surgery* |
| 20 | I + N | R | Lymph node | X | X |
| 5 | N | NR | Lymph node | X | X |
| 7 | N | NR | Lymph node | X | X |
| 9 | N | NR | Lymph node | On Treatment* | Surgery* |
| 16 | N | NR | Lymph node | X | Surgery* |
| 11 | N | NR | Lymph node | X | insufficient |
| 12 | N | NR | Subcut. nodule | X | insufficient |
| 18 | N | NR | Subcut. nodule | On Treatment* | insufficient |
| 21 | N | NR | Subcut, nodule | X | insufficient |
| 22 | N | NR | Lymph node | X | insufficient |
| 3 | I + N | NR | Lymph node | On Treatment* | Baseline* |
| 8 | I + N | NR | Lymph node | Baseilne* | insufficient |
| 14 | I + N | NR | Subcut. nodule | X | X |

Tx, tfeatment;
I + N, ipilimumab with nivolurnab;
N, nivoiumab;
R, responder;
NR, non-responder;
LDH, lactate dehydrogenase;
subcut., subcutaneous
*indicates samples run together and included in t5NE plots and phenographs; bold are samples induded; “insufficient” means sample available and analyzed by mass cytometry, but number of B-cells too low for downstream analyses; “x” indicates no sample available or sample inadequate for mass cytometry

### Example 2—Materials and Methods

Patient Cohort(s) and Sample Collection: For the melanoma neoadjuvant cohort (NCT02519322), 23 patients enrolled in a phase II clinical trial of neoadjuvant ICB. Twelve patients received nivolumab monotherapy with 3 mg/kg every 2 weeks for up to 4 doses, and 11 patients received ipilimumab 3 mg/kg with nivolumab 1 mg/kg every 3 weeks for up to 3 doses followed by surgical resection. These patients were treated at the University of Texas MD Anderson Cancer Center and had tumor samples collected and analyzed under Institutional Review Board (IRB)-approved protocols (2015-0041, 2012-0846). Of note, these studies were conducted in accordance with the Declaration of Helsinski and approved by the UT MD Anderson Cancer Center IRB. Response was defined as achieving a complete or partial radiographic response by RECIST 1.1 between pre-treatment imaging and post-neoadjuvant treatment imaging prior to surgical resection. Tumor samples were collected at several time-points for correlative studies including baseline and on-treatment (weeks 3 and 5 for nivolumab monotherapy, weeks 4 and 7 for combination ipilimumab with nivolumab). Tumor samples were obtained as core, punch or excisional biopsies performed by treating clinicians or an interventional radiologist. Samples were immediately formalin fixed and paraffin-embedded (FFPE), snap frozen, or digested following tissue collection.

Additional patients off-protocol included 5 patients with widely metastatic melanoma who were treated at the University of Texas MD Anderson Cancer Center and had tumor samples collected and analyzed under Institutional Review Board (IRB)-approved protocols (LAB00-063 and PA17-0261). Samples were immediately formalin fixed and paraffin-embedded (FFPE) following tissue collection.

The renal cell carcinoma (RCC) trial was an open-label, randomized, pre-surgical/pre-biopsy trial (NCT02210117) whereby adults with metastatic RCC without prior immune checkpoint therapy and anti-VEGF therapy were enrolled and randomized 2:3:2 to receive nivolumab (3 mg/kg q2 wks×3 doses), nivolumab+bevacizumab (3 mg/kg q2 wks×3+10 mg/kg×3) or nivolumab+ipilimumab (3 mg/kg q2 wks×3 1 mg/kg×2), followed by surgery(cytoreductive nephrectomy or metastasectomy), or biopsy at week 8-10, and subsequent nivo maintenance therapy up to 2 years. Response was assessed at 8 weeks and then at ≥12 weeks by RECIST 1.1 criteria. Clinical response data collection is still ongoing at this time. For this current correlative study, response was defined as achieving a complete or partial response. Pre- and post-treatment blood and tumors were obtained for correlative studies by IRB-approved lab protocol PA13-0291. Tumor samples were obtained as core biopsies or surgical resection performed by interventional radiologists or surgeons. Samples were immediately formalin fixed and paraffin-embedded (FFPE) or snap frozen following tissue collection.

Gene Expression Profiling and Analysis

RNA Extraction for Neoadjuvant Melanoma ICB Cohort.

Total RNA was extracted from snap-frozen tumor specimens using the AllPrep DNA/RNA/miRNA Universal Kit (Qiagen) following assessment of tumor content by a pathologist, and macrodissection of tumor bed if required. RNA quality was assessed on an Agilent 2100 Bioanalyzer using the Agilent RNA 6000 Nano Chip with smear analysis to determine DV200 and original RNA concentration. Based on RNA quality, 40-80 ng of total RNA from each sample then underwent library preparation using the Illumina TruSeq RNA Access Library Prep kit according to the manufacturer's protocol. Barcoded libraries were pooled to produce final 10-12 plex pools prior to sequencing on an Illumina NextSeq sequencer using one high-output run per pool of 76 bp paired-end reads, generating 8 fastq files (4 lanes, paired reads) per sample.

RNA-Seq Data Processing and Quality Check.

RNA-seq FASTQ files were first processed through FastQC (v0.11.5), a quality control tool to evaluate the quality of sequencing reads at both the base and read levels. The reads that had 15 contiguous low-quality bases (phred score<20) were removed from the FASTQ files. STAR 2-pass alignment (v2.5.3) (Dobin et al., 2012) was then performed on the filtered FASTQ files with default parameters to generate RNA-seq BAM file for each sequencing event. After that, RNA-SeQC (v1.1.8) (DeLuca et al., 2012) was run on the aligned BAM files to generate a series of RNA-seq related quality control metrics including read counts, coverage, and correlation. A matrix of Spearman correlation coefficients was subsequently generated by RNA-SeQC among all sequencing events. The correlation matrix was carefully reviewed and the sequencing event generated from one library pool that showed poor correlation with other library pools from the same RNA sample were removed before sample-level merging of BAM files.

Gene Expression Quantification and Normalization.

HTSeq-count (v0.9.1) (Anders and Huber, 2015) tool was applied to aligned RNA-seq BAM files to count for each gene how many aligned reads overlap with its exons. The raw read counts generated from HTSeq-count (v0.9.1) were normalized into fragments per kilobase of transcript per million mapped reads (FPKM) using the RNA-seq quantification approach suggested by the bioinformatics team of NCI Genomic Data Commons (GDC). Briefly, FPKM normalizes read count by dividing it by the gene length and the total number of reads mapped to protein-coding genes using a calculation described below:

FPKM=RC_{g}*10⁹−RC_{pc}*L

RC_{g}, number of reads mapped to the gene; RC_{pc}: number of reads mapped to all protein-coding genes; L, length of the gene in base pairs (calculated as the sum of all exons in a gene). The FPKM values were then logₑ-transformed for further downstream processes.

Affymetrix Microarray for RCC.

The Affymetrix microarray data were created using the Affymetrix Clariom™ D Assay (Human). There are 28 available pre-treatment samples from 3 arms: Nivolumab (n=6), Nivolumab+ Bevacizumab (n=14) and Nivolumab+Ipilimumab (n=8). The raw CEL files were normalized using the built-in SST-RMA method of the Affymetrix Transcriptome Analysis Console (TAC, v4.0) software. The cell lineage scores were calculated using the R package MCP-counter algorithm (v. 1.1.0). The Limma R software package (Ritchie et al., 2015) was used to identify DEGs from normalized microarray data for the RCC cohort.

TCGA SKCM and KIRC Data Downloading and Patient Selection.

The normalized RNA-seq expression data of TCGA skin cutaneous melanoma (TCGA-SKCM) and Kidney Renal ClearCell Carcinoma (TCGA-KIRC) was downloaded from NCI Genomic Data Commons (GDC) and the relevant clinical data was downloaded from recent TCGA PanCancer clinical data study (Liu et al., 2018). The information of SKCM genomic subtypes was obtained from the TCGA-SKCM study. To achieve a uniform cohort of patients with Stage III (non-recurrent) melanoma for analysis, an appropriate set of sequential filters were applied: The TCGA-SKCM cohort was filtered to include patients with biospecimen tissue sites that included regional lymph node or regional subcutaneous metastases. Patients presenting with Stage IV disease were excluded. Then, to exclude patients with recurrent Stage III disease, all patients were excluded for whom the number of days from the diagnosis of the primary to the accession date was >90 days. Additionally, for a patient to be included, their tumor must also have had a defined melanoma driver type. Finally, those lacking sufficient gene expression data were eliminated, yielding a final Stage III TCGA-SKCM cohort of n=136. Survival data missing for 9 or 136 samples, so n=127 for overall survival analyses. For TCGA-KIRC, the cases without available expression data were excluded and a total of 526 cases were taken into subsequent analysis.

Identification of Differentially Expressed Genes.

The HTSeq normalized read count data for all expressed coding transcripts was processed by Deseq2 (v3.6) software to identify differentially expressed genes (DEGs) between two response (R versus NR) groups. A cut-off of gene expression fold change of >2 or <0.5 and a FDR q-value of <0.05 was applied to select the most differentially expressed genes. The Limma R software package was used to identify DEGs from normalized microarray data for the RCC cohort.

Deconvolution of the Cellular Composition with MCP-Counter.

The R package software MCP-counter (Becht et al., 2016) was applied to the normalized logₑ-transformed FPKM expression matrix to produce the absolute abundance scores for 8 major immune cell types (CD3⁺ T-cells, CD8⁺ T-cells, cytotoxic lymphocytes, NK cells, B lymphocytes, monocytic lineage cells, myeloid dendritic cells, and neutrophils), endothelial cells, and fibroblasts. The deconvolution profiles were then hierarchically clustered and compared across response and treatment groups.

Pathway Enrichment Analyses.

The network-based pathway enrichment analysis was performed using differentially expressed genes across responder and non-responder groups in the bulk-tissue RNA sequencing data from melanoma neoadjuvant cohort. In the bulk-tissue, the differentially expressed genes which had a q-value<0.05 and log 2 foldchange>1.5 were & <−1.5 were selected as input for network based pathway enrichment analysis using ReactomeFiViz application in Cytoscape. Pathway enrichment was calculated using several biological databases (KEGG, NCBI, Reactome, Biocarta, and Panther) with hypergeometric test false discovery rate (FDR)<0.01.

Survival Analyses.

In TCGA cohort, survival data was not available for 9 samples and these were excluded from survival analysis. As described previously (Cancer Genome Atlas Network, 2015) the survival time for each patient was “Curated TCGA survival (i.e., from time of TCGA biospecimen procurement). The time to event was defined as the time interval from date of accession for each sample to date of death or censoring from any cause (curated value CURATED_TCGA_days_to_death_or_last_follow-up; aka TCGA post-accession survival). The survival analysis was performed using Cox Proportional Hazards model and survival curves were plotted using Kaplan-Meier method. The statistical comparison of the survival curves was done using the log rank test. The analysis was done using R package survival (Therneau and Lumley, 2015).

Statistical Analyses.

The statistical comparison between responder and non-responder groups for a given continuous variable was performed using two-sided Mann-Whitney U test. The association between two continuous variables was assessed using Spearman's rank correlation coefficient. To control for multiple comparisons, the Benjamini-Hochberg method was applied and adjusted P-values were calculated. Univariable and multivariable analysis predicting response to ICB was performed using logistic regression modeling.

Single Immunohistochemistry.

Hematoxylin (H&E) and immunohistochemistry (IHC) staining were performed on FFPE tumor tissue sections. The tumor tissues were fixed in 10% formalin, embedded in paraffin, and transversely sectioned. 4 μm sections were used for the histo-pathological study. Sections were stained with mouse or rabbit anti-human monoclonal antibodies against CD20 442 (Dako, cat # M0755, 1:1400), CD21 (Novocastra, NCL-L-CD21-2G9, 1:10 or Leica, CD21-2G9; 443 1:20), CD23 (Leica, CD23-1B12, 1:15), CD4 (Novocastra, CD4-368-L-A, 1:80) CD8 (Thermo Scientific, MS-457-S, 1:25), FoxP3 (Biolegend, Cat #320102, 1:50). All sections were counterstained with hematoxylin, dehydrated, and mounted. All sections were processed with peroxidase-conjugated avidin/biotin and 3′-3-diaminobenzidine (DAB) substrate (Leica Microsystem) and slides were scanned and digitalized using the scanscope system from Scanscope XT, Aperio/Leica Technologies.

Quantitative analysis of IHC staining was conducted using the image analysis software ImageScope-Aperio/Leica. Five random areas (1 mm² each) were selected using a customized algorithm for each marker in order to determine the number of positive cells at high power field (HPF). The data is expressed as a density (total number of positive cells/mm² area). IHC staining was interpreted in conjunction with H&E stained sections.

Tertiary Lymphoid Structure Quantification.

Tertiary lymphoid structures (TLS) were qualified and quantified using both H&E and CD20 IHC staining. Structures were identified as aggregates of lymphocytes having histologic features with analogous structures to that of lymphoid tissue with follicles, appearing in the tumor area (Dieu-Nosjean et al., 2014). For the current study, criteria used for the quantification of TLS includes: 1) the total number of structures identified either within the tumoral area or in direct contact with the tumoral cells on the margin of the tumors (numbers of TLS/mm² area); and 2) a normalization of the total area occupied by the TLNs in relation of the total area of the tumor analyzed (ratio: area of TLS/area tumor+TLNs).

Multiplex Immunofluorescence Assay and Analysis.

(FIG. 4, 8, 9) For IF multiplex staining, the staining method was followed for the following markers: CD20 (Dako, cat # M0755, 1:500) with subsequent visualization using fluorescein Cy3 (1:50); CD21 (Novocastra, NCL-L-CD21-2G9, 1:10) with subsequent visualization using fluorescein Cy5 (1:50); CD4 (CM153BK, Biocare, 1:25) with subsequent visualization using fluorescein Cy5.5 (1:50); CD8 (1:200, M7103, Dako) with subsequent visualization using fluorescein Cy3.5 (1:50); FoxP3 (Biolegend, Cat #320102, 1:50) with subsequent visualization using fluorescein FITC (1:50) and nuclei visualized with DAPI (1:2000). All of the sections were cover-slipped using Vectashield Hardset 895 mounting media. The slides were scanned using the Vectra slide scanner (PerkinElmer). For each marker, the mean fluorescent intensity per case was then determined as a base point from which positive calls could be established. For multispectral analysis, each of the individually stained sections was utilized to establish the spectral library of the fluorophores. Five random areas on each sample were analyzed blindly by a pathologist at 20× magnification.

B-Cell Clonotype Analyses

The Modified TRUST Algorithm (Hu et al., 2019) was applied to extract the B-cell immunoglobin hypervariable regions from the bulk RNA—seq data and assembly the complementarity-determining region 3 (CDR3) sequences of the B-cell heavy chain (IgH) and light chain (IgL). BCR clonotypes were identified and the clonal fraction was automatically calculated by TRUST. The output of TRUST was parsed by the R package tcR (version 3.4.1) for downstream analyses. Only in-frame productive clonotypes were taken into subsequent analysis. The total number of BCR clonotypes detected per sample was normalized by the corresponding sequencing depth of each individual sample and calculated as per 100 million mapped reads. The top 5 clonotypes were selected by their clonal expression abundance. The BCR repertoire diversity was calculated by entropy from the tcR package.

Details for Mass Cytometry (CyTOF)

Antibody Conjugation.

In-depth characterization of R and NR B-cells was performed using metal-tagged antibodies. Metal conjugated antibodies were purchased from Fluidigm or conjugated to unlabeled antibodies in-house. All unlabeled antibodies were purchased in carrier-free form and conjugated with the corresponding metal tag using Maxpar X8 polymer per manufacturer's instructions (Fluidigm). Metal isotopes were acquired from Fluidigm and indium (III) chloride was acquired from Sigma-Aldrich. Antibody concentration was determined by measuring the amount of A280 protein using Nanodrop 2000 (Thermo Fisher Scientific). Conjugated antibodies were diluted using PBS-based antibody stabilizer supplemented with 0.05% sodium azide (Sigma-Aldrich) to a final concentration of 0.5 mg/ml. The list of antibodies with the corresponding metal tag isotopes is shown in table below.

TABLE 11
Antibodies.

| TARGET | Clone | ISOTOPE | Source |
|---|---|---|---|
| CD45 | HI30 | 89y | Fluidigm |
| CD80 | 2D10 | 115in | Biolegend |
| CD138 | MI15 | 141pr | BD Biosciences |
| CD19 | HIB19 | 142Nd | Fluidigm |
| CD5 | UCHT2 | 143Nd | Fluidigm |
| HLA-ABC | EMR8-5 | 144Nd | BD Biosciences |
| CD178 | NOK-1 | 145Nd | Biolegend |
| IgD | IA6-2 | 146Nd | Biolegend |
| CD20 | 2H7 | 147sm | Fluidigm |
| PDL-1 | 29E.2A3 | 148Nd | Fluidigm |
| HLA-DR | L243 | 149sm | Biolegend |
| CD25 | 2A3 | 150Nd | BD Biosciences |
| IGM | MHM-88 | 151Eu | Biolegend |
| CD95 | DX2 | 152sm | BD Biosciences |
| CXCR5 | RF8B2 | 153Eu | Fluidigm |
| CD86 | IT2.2 | 154sm | BD Biosciences |
| CD27 | L128 | 155Gd | Fluidigm |
| CXCR3 | G025H7 | 156Gd | Biolegend |
| CD10 | HI10a | 158Gd | Fluidigm |
| PDL-2 | 24F.10C12 | 159Tb | Biolegend |
| CD39 | Al | 160Gd | Fluidigm |
| BAFF-R | 11C1 | 161Dv | Biolegend |
| CD79b | CB3.1 | 162Dv | Fluidigm |
| CD1d | 51.1 | 163Dv | Biolegend |
| CD23 | EBVCS-5 | 164Dv | Fluidigm |
| CD40 | 5C3 | 165H0 | Biolegend |
| CD24 | ML5 | 166Er | BD Biosciences |
| CD38 | HIT2 | 167Er | BD Bioscience |
| CD21 | Bu32 | 168Er | Biolegend |
| ICOS | C398.4A | 169Tb | Biolegend |
| CTLA-4 | 14D3 | 170Er | Fluidigm |
| CD9 | HI9a | 171yb | Biolegend |
| CD1 lc | Bul5 | 172yb | Biolegend |
| CD14 | HCD14 | 173yb | Biolegend |
| PD1 | PD1.3.1.3 | 174yb | Miltenyi |
| CXCR4 | 12G5 | 175Lu | Biolegend |
| CD22 | HIB22 | 176yb | Biolegend |
| CD3 | UCHT-1 | 194p | Biolegend |
| Cisplatin | | 198pt | Fluidigm |
| CD16 | 3G8 | 209Bi | Fluidigm |

Sample Preparation and Acquisition.

Peripheral blood mononuclear cells (PBMCs) and tumor cells were harvested and washed twice with wash buffer (0.5% bovine serum albumin (BSA) in PBS). For tumor, this included 9 R and 9 NR, and for PBMCs, 8 R and 8 NR. To determine the live population, cells were stained with cisplatin 1 μM for 3 minutes. The reaction was stopped with FACS buffer (2% Fetal Bovine Serum (FBS) in PBS), and the cells were washed once with wash buffer. Cells were then incubated with 5 μl of Fc receptor blocking buffer reagent (Miltenyi) for 10 minutes at room temperature. Cells were incubated with surface antibodies at room temperature for 60 minutes, washed twice with wash buffer and stored overnight in 1 ml of 1.6% paraformaldehyde (EMD Biosciences) in PBS with 125 nM iridium nucleic acid intercalator (Fluidigm). The next day, samples were washed twice with cell staining buffer, re-suspended in 1 ml of MilliQ dH2O, filtered through a 35 μm nylon mesh (cell strainer cap tubes, BD, San Jose, Calif.) and counted. Before analysis, samples were resuspended in MilliQ dH₂O supplemented with EQ™ four element calibration beads at a concentration of 0.5×10⁵/ml. Samples were acquired at 300 events/second on a Helios instrument (Fluidigm) using the Helios 6.5.358 acquisition software (Fluidigm).

Data Analysis.

Mass cytometry data were normalized based on EQ™ four element signal shift over time using Fluidigm normalization software 2. Initial data processing was performed using Flowjo version 10.2. Mass cytometry data were normalized based on EQ™ four element signal shift over time using Fluidigm normalization software 2. Initially, all R and NR normalized FCS files were either concatenated or separately exported for downstream analyses. Data were processed and analyzed using Cytobank; CD19⁺ sample ‘clean-up’ was performed by gating on intact (¹⁹¹Ir⁺ DNA stain), no beads (¹⁴⁰Ce⁻), live (¹⁹⁸Pt⁻), no T-cells CD3 (¹⁹⁴Pt), no monocytes

CD14(₁₇₃Yb) and CD45₊(₈₉Y), no NK Cells CD16₋(₂₀₉Bi). CD19₊ B-cells. Mass cytometry complex data were analyzed using viSNE, in combination with heat map, to identify distinct subpopulations using the following parameters: CD19(₁₄₂Nd), CD20(₁₄₇Sm), CD5(₁₄₃Nd), HLA-ABC(₁₄₄Nd), IgD(₁₄₆Nd), PDL-1(₁₄₈Nd), HLA-DR(₁₄₉Sm), CD25(₁₅₀Nd), IgM(₁₅₁Eu), CD95(₁₅₂Sm), CXCR5(₁₅₃Eu), CD86(₁₅₄Sm), CD27(₁₅₅Gd), CXCR3(₁₅₆Gd), CD10(₁₅₈Gd), CD39(₁₆₀Gd), BAFFR(₁₆₁Dy), CD79b(₁₆₂Dy), CD1d(₁₆₃Dy), CD23(₁₆₄Dy), CD40(₁₆₅Ho), CD24(₁₆₅Er), CD38(₁₆₇Er), CD9(₁₇₁Yb), CD11c(₁₇₂Yb), CXCR4(175Lu), and CD22(176Yb). Samples with less than 200 CD45+CD19+ B− cells were not utilized for downstream analyses. Percentages of different sub-populations of B-cells were measured in aggregated R and NR PBMC and tumor samples for each run; statistical analyses performed via unpaired Student's t-test.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Amaria, et al. Nature Medicine, 2018.
Anders & Huber, Bioinformatics 31, 166-169, 2015.
Becht, et al. Genome Biol 17, 218, 2016.
Blank, et al. Nat Med 24, 1655-1661, 2018.
CancerGenomeAtlasNetwork. Cell 161, 1681-1696, 2015.
Chen, et al. Cancer Discov 6, 827-837, 2016.
DeLuca, et al. Bioinformatics 28, 1530-1532, 2012.
Dieu-Nosjean, et al. Trends Immunol 35, 571-580, 2014.
Dobin, et al. Bioinformatics 29, 15-21, 844 2013.
Hu, et al. Nat Genet 51, 560-567, 2019.
Liu, et al. Cell 173, 400-416 e411, 2018.
Posch, et al. Oncoimmunology 7, e1378844, 2018.
Ritchie, et al. Nucleic Acids Res 43, e47, 2015.
Sade-Feldman, et al. Cell 175, 998-1013 e1020, 2018.
Therneau & Lumley, R Top Doc 128, 2015.
Villani, et al. Science 356, 2017.

1. A method of treating cancer in a subject comprising administering an immune checkpoint blockade (ICB) therapy to the subject, wherein the subject has been determined to have a B cell signature.

2. The method of claim 1, wherein the subject has a low percentage of tumor-infiltrating CD8⁺ T cells as dichotomized at a median value, wherein the low percentage is a CD8 T cell score of less than 0 on microenvironment cell population (MCP) counter performed on gene expression profiling.

3. (canceled)

4. The method of claim 1, wherein the B cell signature comprises a high density of tumor-infiltrating B cells and/or high chemokine C-X-C motif ligand 13 (CXCL13) expression.

5. The method of claim 4, wherein the high density is further defined as a B cell lineage score of greater than −0.40 on MCP counter performed on gene expression profiling.

6. The method of claim 4, wherein the subject has a tumor-infiltrating B cell density of at least 500 cells/mm² in the tumor.

7-10. (canceled)

11. The method of claim 1, wherein the subject has a tumor comprising tertiary lymphoid structures (TLS).

12. The method of claim 11, wherein the subject has a TLS density of at least 0.5 TLS/mm² in the tumor or a ratio of at least 0.25 TLS per tumor area.

13-16. (canceled)

17. The method of claim 1, wherein the B cell signature comprises increased expression of B cell scaffold protein with ankyrin repeats 1 (BANK1), B lymphocyte antigen cluster of differentiation 19 (CD19), cluster of differentiation CD2 (CD22), cluster of differentiation 79A (CD79A), complement receptor type 2 (CR2), Fc receptor-like protein 2 (FCRL2), immunoglobulin kappa constant (IGKC), B-lymphocyte antigen CD20 (MS4A1), and/or paired box protein PAX-5 (PAX5).

18-25. (canceled)

26. The method of claim 1, wherein the B cell signature comprises co-localization of tumor-infiltrating B cells with CD4⁺, CD8⁺, FoxP3⁺ T lymphocytes, and/or CD21 follicular dendritic cells.

27. (canceled)

28. The method of claim 1, wherein the cancer is melanoma.

29-35. (canceled)

36. The method of claim 1, wherein the ICB therapy comprises one or more inhibitors of cytotoxic T lymphocyte associated protein 4 (CTLA-4), programmed cell death protein 1 (PD-1), programmed death ligand 1 (PD-L1), programmed death ligand 2 (PD-L2), lymphocyte activation gene 3 (LAG-3), B and T lymphocyte attenuator (BTLA), cluster of differentiation 276 (B7H3), V set domain containing T cell activation inhibitor 1 (B7H4), T cell immunoglobulin and mucin domain 3 (TIM3), killer cell immunoglobulin-like receptor (KIRK, or adenosine A2A receptor (A2aR).

37. The method of claim 1, wherein the ICB therapy comprises an anti-PD1 antibody and/or an anti-CTLA4 antibody.

38. The method of claim 37, wherein the anti-PD1 antibody is nivolumab, pembrolizumab, pidillizumab, KEYTRUDA®, AMP-514, REGN2810, CT-011, BMS 936559, MPDL328OA or AMP-224.

39. The method of claim 37, wherein the anti-CTLA-4 antibody is tremelimumab, YERVOY®, or ipilimumab.

40. The method of claim 37, wherein the subject is administered nivolumab at a dose of 1 mg/kg and/or is administered ipilimumab at a dose of 3 mg/kg.

41. (canceled)

42. The method of claim 1, wherein the subject is further administered or has been administered an anti-VEGF therapy.

43. (canceled)

44. The method of claim 1, further comprising administering at least one additional anti-cancer therapy.

45. The method of claim 44, wherein the anti-cancer therapy is a lymphotoxin receptor beta agonist or a CD40 agonist.

46-50. (canceled)

51. An in vitro method for detecting a B cell signature in a sample comprising:
(a) obtaining a tumor sample from a subject diagnosed with cancer; and
(b) detecting tumor-infiltrating B cells and/or TLS in said tumor sample, wherein an increased level of tumor-infiltrating B cells and/or TLS as compared to a control detects the B cell signature.

52-64. (canceled)

65. A method for treating cancer in a subject comprising administering ICB therapy to the subject, wherein the subject has been determined to have a tumor with TLS.

66-106. (canceled)
